(19) United States
(12) Patent Application Publication
(10) Pub. No.: US 2006/0039986 A1
(43) Pub. Date: Feb. 23, 2006

(54) Functional material, process for producing functional material and functional member and environment modifying apparatus using the functional material

(75) Inventors: Hiroshi Okamoto, Owari-asahi-shi (JP); Shin-ichi Inoue, Tokoname-shi (JP); Masataka Sano, Hamamatsu-shi (JP); Yoko Nagai, Hamamatsu-shi (JP); Hiroki Miyamatsu, Hamamatsu-shi (JP); Kimi Yoshida, Hamamatsu-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kabushiki Kaisha Erubu, Hamamatsu-shi (JP)

(21) Appl. No.: 11/167,879

(22) Filed: Jun. 28, 2005

(30) Foreign Application Priority Data
Jul. 1, 2004 (JP) 2004-195307
Jul. 1, 2004 (JP) 2004-195310
Jul. 1, 2004 (JP) 2004-195315
Jan. 28, 2005 (JP) 2005-22316

(51) Int. Cl. A61K 9/14

(52) U.S. Cl. 424/489

(57) ABSTRACT

A functional material of the present invention is characterized in that it has: a loading component selected from functional components comprising the group consisting of catechins, vitamins, tannins, natural moisturizing factors and essential oils being derived from plants; and a functional ingredient being constituted of an organic polymer material in which the loading component is loaded on the surface or in the inside thereof, and which is a fine particle; and it exhibits dispersibility with respect to linseed oil. Since the functional material comprising the functional ingredient with the functional component loaded exhibits high dispersibility with respect to the oil, it turns out that it is possible to provide a functional material which exhibits good properties as fragrant cosmetics of good stability. In particular, a function material prepared by means of a spray-drying method is preferable.

### BACKGROUND OF THE INVENTION

1. Field of the Invention

The present invention relates to a functional material, which exhibits a functionality, such as an anti-microorganism property, a deodorizing property, an anti-oxidation property, a moisturizing property, a relaxation property, a freshness retention characteristic, a whitening effect and an anti-inflammation effect, and a process for producing a functional material, as well as a functional material and an environment modifying apparatus, using the functional material.

2. Description of the Related Art

Substrates, such as fibers, films and various component parts, and coating compositions, such as paints and cosmetics, have been in widespread use in all fields, beginning with industrial applications, consumer applications, medical applications and agricultural applications. And, recently, for the purpose of the improvement of living environments, it has been often the case to give various functionalities, such as anti-microorganism properties and deodorizing properties, to these substrates and coating compositions.

Moreover, due to the recently growing health consciousness, or for the purpose of the improvement of living environments, developments have been underway with a keyword, such as the improvement of amenity to human beings, in mind in various home electric appliances, and the like, which have been used conventionally, as well. For example, it has been carried out to employ filters, on which functional components demonstrating functionalities are loaded, for air conditioners. As for the functional components, synthetic medical agents are also effective, however, it is recommended to use effective components being derived from natural products, considering the safety.

When giving various functionalities, such as anti-microorganism properties and deodorizing properties, to the substrates and coating compositions, it has been carried out to contain or load medical agents being capable of demonstrating those functionalities. As for the medical agents, synthetic medical agents are also effective, however, it is recommended to use effective components being derived from natural products, considering the safety.

From this viewpoint, the present applicants have been carrying out quite a few number of patent applications on the techniques for giving functionalities to the substrates and coating compositions using extracts (tea-leave extracts, catechin, saponin, and the like), and so forth, being derived from tea.

For example, in Japanese Unexamined Patent Publication (KOKAI) No. 2000-204,277 according to one of the present applicants' applications, there is set forth a functional molded substance comprising a molten moldable substance in which a functional component exhibiting an anti-microorganism property or a deodorizing property, the functional component being selected from the group consisting of catechins, saponins, tea-leave powders, tea-leave extracts and tannin (acid), and a ceramic component are compounded.

Moreover, in Japanese Unexamined Patent Publication (KOKAI) No. 2002-316,909, the present applicants' another patent application, there is set forth a functional material being characterized in that it comprises at least one member of effective components (B) selected from the group consisting of essential oils or extracts being derived from tea trees, pine, clove, sage, nutmeg, gingko leaves, rice hull, leeks, Galanga, Kaffir-lime (citrus-hystrix), coffee beans, guava tea, checker tree, lithospermum root, bamboo, or “kumazasa” bamboo; glycoside being derived from western mustard; polysaccharide being derived from hyaluronic acid or Agaricus fungus; protein being derived from plants and animals or microorganisms, or its decomposed products; amino acid or its derivative; rice-malt acid or red-rice-malt decomposed products; ascorbic acid, vitamin D; caffeine; and a ceramic component (C).

Further, in Japanese Unexamined Patent Publication (KOKAI) No. 2003-235,948 according to one of the present applicants' applications, there is set forth a production method for obtaining a fine-particulate hybridized substance, wherein an aqueous slurry (C), containing an organic component (A) exhibiting a functionality and a ceramic component (B), and a spray-drying apparatus are used to hybridize the organic component (A) and ceramic component (B)

Moreover, in Japanese Unexamined Patent Publication (KOKAI) No. 6-72,849, there is a disclosure on a whitening cosmetic containing L-ascorbic acid or its water-soluble derivative and a tea-leave-extract content, and there is a description that a melanin-generation inhibition action can be obtained by the combined use of the aforementioned components. Moreover, there is Japanese Unexamined Patent Publication (KOKAI) No. 2002-53,416 as a relevant application.

Furthermore, in Fragrance journal 1997.1.p87-90, there is a description on ascorbil glucosamine, and there are advocated advantages, such as the improvement of anti-oxidation performance and the improvement of collagen-production facilitation ability, compared with ascorbic acid, moreover the anti-oxidation property and anti-collagenase activity which are held longer.

### SUMMARY OF THE INVENTION

(Assignment to be Solved by the Invention)

However, as disclosed in the aforementioned literatures, by simply loading effective components being derived from natural products on inorganic components, or by simply adhering them on or adding them internally in physical objects, such as fibers, there have been cases where sufficient effects cannot be demonstrated. For example, the following have occurred: the functionalities due to effective components cannot necessarily be demonstrated sufficiently; acridity has been too intense because the volatility is excessive; effective components have been lost easily by evaporation or elution; and when being adhered on physical objects or being added internally in them, they impair the texture, feel, strength, and the like, of their physical objects. Further, it has occurred that effective components themselves are very likely to decompose so that they do not arrive at demonstrating their functionalities with good stability. In particular, ascorbic acid is likely to decompose. Moreover, the volatile substances of various components are such that their vaporizing amounts increase by applying heat thereto.

Therefore, regarding the production processes of functional materials, they are expected to be carried out without applying temperature, if possible, however, in order to stably load a functional component on ceramic particles, and the like, which the present inventors have investigated conventionally, a temperature of 170° C. or more is needed, for example, when using colloidal silica. It has been desired that a process for stably loading effective components, which are likely to be decomposed by heat, at a much lower temperature.

Moreover, for the purpose of further improving living environments, it has been required to give further functionalities, such as anti-allergic actions, moisturizing actions, actions contributing to health or beautification, environmental-atmosphere improving actions, relaxation actions, aroma-therapeutic actions, freshness retaining actions and anti-growing actions, in such a state that high effect and stability are made compatible, and their material developments have been desired.

Moreover, for the purpose of further improving living environments, it has been required to give functionalities, such as anti-allergic actions, moisturizing actions, actions contributing to health or beautification, environmental-atmosphere improving actions, relaxation actions, aroma-therapeutic actions, freshness retaining actions and anti-growing actions, in such a state that high effect and stability are made compatible, and their material developments have been desired.

The present invention is one which has been done in view of the aforementioned circumstances, and an assignment to be solved is, in order to respond to the market's requirement of performance improvement, to provide a functional material capable of demonstrating higher stability than conventionally, a process for producing a functional material as well as a functional member using the functional material, and an environment modifying apparatus using the functional member as a filter.

(Means for Solving the Assignment)

(First Means)

As a result of carrying out investigation earnestly, the present investors understood that it is possible to provide a material which is good as fragrant cosmetics when a functional material comprising a functional ingredient, on which a functional component is loaded, exhibits high dispersibility with respect to oil. The present inventors completed the present invention based on the aforementioned knowledge.

A functional material of the present invention, which solves the aforementioned assignment, exhibits dispersibility with respect to linseed oil, and is characterized in that it has: a loading component selected from functional components comprising the group consisting of catechins, vitamins, tannins, natural moisturizing factors and essential oils being derived from plants; and
a functional ingredient being constituted of an organic polymer material in which the loading component is loaded on the surface or in the inside thereof, and which is a fine particle.

Here, “exhibiting dispersibility with respect to linseed oil” refers to that no separation occurs visually for 4 hours or more after 0.2-g present functional material is suspended with respect to 20-mL linseed oil. “Separation occurs” refers to that a boundary line arises between the portions in which the linseed oil and present functional material are suspended.

(Second Means)

Further, the present inventors found out that it is possible to provide a functional material of high stability which meets the respective objects, when using two types or more of functional components, and a functional ingredient. Moreover, by designing the mixing ratio of the two types or more of functional components so as to meet objective applications by means of selecting the type of the functional ingredient, controlling the production conditions of the functional material, and the like, they found out that it is possible to provide a functional material which meets the respective objects. The present inventor completed the present invention based on the aforementioned knowledge.

Specifically, a functional material of the present invention, which solves the aforementioned assignment, is characterized in that it has: two or more loading components selected from functional components comprising the group consisting of catechins, vitamins, tannins, natural moisturizing factors and essential oils being derived from plants, and at least a part thereof interacting with each other; and a functional ingredient in which the loading component is loaded on the surface or in the inside thereof, and which is a fine particle.

That is, by employing two or more functional components as the loading components and interacting each of the loading components or at least a part of them mutually, it is possible to stably load the loading components on the functional ingredient. Here, “interacting them with each other” means that they are turned into a physically and/or chemically bonded state. The loading components and functional ingredient can desirably be selected from materials which have chemical structures of high reactivity. As for the chemical structures of high reactivity, an OH group can be exemplified, and materials having OH groups on the surface are preferable. The OH groups are condensed by dehydration under high temperatures, and can realize a firm bond by means of hydrogen bond, and the like, as OH group.

When the functional components exist independently, many of them are unstable, and it is necessary to stabilize them by derivatization, and the like. It was found out that the functional components can exist in a stabilized manner by loading them on the functional ingredient, moreover, they are further stabilized by mixing two or more types of them. Moreover, it becomes possible to further give functionalities by loading two or more types of the functional components.

Moreover, when making compounds by reacting two or more types of the functional components alone, it is believed that the molar ratios of different components' compounds are fixed by the reactivities of the components so that the components, which have not took part in the reaction, are removed. However, by loading them on the functional ingredient, the functional ingredient assumes a binder form so that it is possible to think of a form in which the components react with each other. Therefore, it is possible to arbitrarily design the ratios of different components in compliance with objective applications, depending on the conditions of the functional ingredient on which they are loaded.

In particular, it is often the case that the functional components being derived from natural products become various mixtures, and they are likely to be decomposed thermally with ease. When being decomposed thermally, it is difficult to maintain the inherent efficacy of natural products. By using said functional ingredient, it is possible to produce the functional material at a lower temperature than conventionally. Accordingly, it is possible to provide the functional material which depresses the decomposition of the functional components and maintains the inherent efficacy possessed by natural products more.

Moreover, by using said functional ingredient, the texture of the completed functional material gets silky, and the touch is smooth as well.

(Third Means)

And, as for another functional material of the present invention which solves the aforementioned assignment, it is characterized in that it is producible by a dropletizing step of turning an aqueous slurry, having two or more loading components selected from functional components comprising the group consisting of catechins, vitamins, tannins, natural moisturizing factors and essential oils being derived from plants and a functional ingredient which is a fine particle, into a fine droplet state; and a drying step of drying the fine droplet by contacting it with a hot air.

That is, the functional material having high stability can be obtained by first mixing the loading components with the functional ingredient to make an aqueous slurry, dropletizing them in such a state that they make the aqueous slurry, and hot-air drying them. As for the reason therefor, it is assumed that firm bonds arise between the loading components or between the loading components and the functional ingredient.

(Fourth Means)

Further, a process for producing the functional material of the present invention which solves the aforementioned assignment is characterized in that it has: a dropletizing step of turning an aqueous slurry having two or more loading components selected from functional components comprising the group consisting of catechins, vitamins, tannins, natural moisturizing factors and essential oils being derived from plants, and a functional ingredient being a fine particle into a fine droplet state; and a drying step of drying the fine droplet by contacting it with a hot air.

(Fifth Means)

A functional member of the present invention which solves the aforementioned assignment is characterized in that it has: two or more loading components selected from functional components comprising the group consisting of catechins, vitamins, tannins, natural moisturizing factors and essential oils, and at least a part thereof interacting with each other; and a support loading the loading components thereon or containing them therein, and being capable of letting air pass in at least one direction.

That is, by employing two or more functional components as the loading components and interacting each of the loading components or at least a part of them mutually, it is possible to stably load the loading components on the functional ingredient. Here, “interacting them with each other” means that they are turned into a physically and/or chemically bonded state. The loading components and functional ingredient can desirably be selected from materials which have chemical structures of high reactivity. As for the chemical structures of high reactivity, an OH group can be exemplified, and materials having OH groups on the surface are preferable. The OH groups are condensed by dehydration under high temperatures, and can realize a firm bond by means of hydrogen bond, and the like, as OH group.

And, when including ascorbic acid as the loading component, it is preferable to control the pH of the functional member toward the acidic side in view of the stability improvement. The pH control can be carried out by adding a pH-adjusting agent. From the results of later-described examples, it is revealed that controlling the pH to 5 or less particularly, further, 3 or less, is preferable. Here, “the pH of the functional member” is a pH when the surface of the functional member or the inside thereof is brought into contact with water.

Further, an environment modifying apparatus of the present invention which solves the aforementioned assignment is characterized in that it has a filter being the above-described functional member, air-sending-out means for letting air pass through the filter, and moisturizing means for moisturizing the air passing through the filter.

The emission amount of the loading component contained in the filter can be controlled by adjusting the humidity of air.

(Sixth Means)

Further, as a result of carrying out investigation earnestly, the present investors can suppress the production temperature lower than conventionally by using a functional ingredient selected from the group consisting of cellulose, cellulose derivatives and polyvinyl alcohol. By making it possible to lower the production temperature, the destruction and evaporation of said functional component by heat can be depressed more so that it has become likely to demonstrate the functionality. Moreover, by changing the mixing ratio of said functional ingredient containing the functional component or being loaded therewith with said ceramic fine particles, they found out that it is possible to design starting from those which emit said functional component by small amount and have longer lives to those which have shorter lives but can emit said functional component in higher concentrations, in compliance with objects.

That is, a functional material of the present invention which solves the aforementioned assignment is characterized in that it has: ceramic particles; a functional ingredient selected from the group consisting of cellulose, cellulose derivatives and polyvinyl alcohol, and adhering on a surface of said ceramic particles or covering it; a functional component selected from the group consisting of catechins, vitamins, tannins, natural moisturizing factors and essential oils being derived from plants, and contained inside said functional ingredient or loaded on a surface thereof.

By using said functional ingredient selected from the group consisting of cellulose, cellulose derivatives and polyvinyl alcohol, it has become possible to provide said functional material on which said functional component, which is likely to decompose or evaporate by heat, is loaded stably.

By changing the compounding ratio of said ceramic particles with said functional ingredient, it is possible to alter the amount of the functional ingredient for adhering on the ceramic particles or being covered therewith. Thus, it is possible to arbitrarily design the releasability of the functional component contained in or loaded on said functional ingredient, in compliance with objects.

(Seventh Means)

And, as for another functional material of the present invention which solves the aforementioned assignment, it is characterized in that it is producible by a dropletizing step of turning an aqueous slurry into a fine droplet state, the aqueous slurry having: ceramic particles; a functional ingredient selected from the group consisting of cellulose, cellulose derivatives and polyvinyl alcohol; and a functional component selected from the group consisting of catechins, vitamins, tannins, natural moisturizing factors and essential oils being derived from plants; and a drying step of drying said fine droplet by contacting it with a hot air.

The functional material having high stability can be obtained by dropletizing said functional component, said functional ingredient and said ceramic particles in such a state that they are mixed to make the aqueous slurry, and hot-air drying them at a low temperature. As for the reason thereof, it is assumed that said functional ingredient is such that the affinity to said functional component and said ceramic particles is high. Moreover, by using said functional ingredient being an organic substance, they can be dried even when the hot-air temperature is lowered more than conventionally. Since it can be produced at a low temperature, it is possible to provide said functional material without decomposing said functional component as much as conventionally.

(Eighth Means)

Further, a process for producing a functional material of the present invention which can solves the aforementioned assignment is characterized in that it has it has: a dropletizing step of turning an aqueous slurry into a fine droplet state, the aqueous slurry having: ceramic particles; a functional ingredient selected from the group consisting of cellulose, cellulose derivatives and polyvinyl alcohol; and a functional component selected from the group consisting of catechins, vitamins, tannins, natural moisturizing factors and essential oils being derived from plants; and a drying step of drying said fine droplet by contacting it with a hot air.

### BRIEF DESCRIPTION OF THE DRAWINGS

FIG. 1 is an explanatory diagram for illustrating a spray-drying apparatus used in examples of the present invention.

FIG. 2 illustrates the ESR measurement results of an example of the present invention and comparative examples.

FIG. 3 illustrates the thermal-analysis measurement results for comparing Example No. 1, Comparative Example No. 1 and Comparative Example No. 2-1.

FIG. 4 illustrates the thermal-analysis measurement results for comparing Example No. 2-2, Comparative Example No. 2-2 and Comparative Example No. 9-2.

FIG. 5 illustrates the SEM observation results of Example No 1.

FIG. 6 illustrates the SEM observation results of Comparative Example No 1.

FIG. 7 illustrates the SEM observation results of Comparative Example No 2-1.

FIG. 8 illustrates the anti-oxidation-ability measurement results of an example of the present invention and comparative examples.

FIG. 9 illustrates the thermal-analysis measurement results for comparing Example Nos. 11 and 12, Comparative Example No. 9 and Comparative Example No. 12.

FIG. 10 is a graph for illustrating the measurement results of the anti-oxidation abilities of an example in examples and comparative examples.

FIG. 11 is a graph for illustrating the moisture dependency of the functional-component emission amount in an example.

FIG. 12 is graphs for illustrating the pH dependency of the stability of a functional component.

FIG. 13 illustrates the SEM observation result of Test Example No. 11.

FIG. 14 illustrates the SEM observation result of Test Example No. 5.

FIG. 15 illustrates the SEM observation result of Test Example No. 6.

### BEST MODE FOR CARRYING OUT THE INVENTION

(Functional Material #1 of First Embodiment Mode)

The functional material of the present invention has two types or more of loading components, and a functional ingredient. The loading components are selected from functional components comprising the group consisting of catechins, vitamins, tannins, natural moisturizing factors and essential oils being derived from plants. At least a part of the loading components are reaction products which interact with each other. The functional ingredient is a fine particle, and the loading components are loaded on the surface or in the inside thereof. As for the interactive action, it is a concept that includes a state in which voids are formed by one of them and the other one of them are captured therein, in addition to physical and/or chemical bonds, for example, the general covalent bonds, hydrogen bonds, and bonds due to van der Waals forces. Between the loading components and the functional ingredient as well, it is desirable that they interact with each other.

In particular, the two types or more of loading components and the functional ingredient being a fine particle are such that a part of them can preferably bond chemically between the loading components, or between the loading components and the functional material. As a result, it is possible to stably load the loading components on the functional ingredient. In particular, by employing two types or more of functional components, it is revealed that the stabilizing effect is high. The loading components and functional ingredient can desirably be selected from materials which have chemical structures of high reactivity. As for the chemical structures of high reactivity, an OH group can be exemplified, and materials having OH groups on the surface are preferable. The OH groups are condensed by dehydration under high temperatures, or can realize a firm bond by means of hydrogen bond, and the like, as OH group.

The application of the functional material is useful for a variety of applications beginning with cosmetic materials, food materials, health food materials, filter materials for air conditioners, air cleaners, cleaners, and the like, clothes, bedclothes-related materials, hygienic materials, footwear materials, carpet materials, kitchen utensils, toiletry utensils, interior materials for buildings or vehicles, building materials, medical materials, agricultural or gardening materials, and packaging materials.

The functional material can preferably be a fine particle. For example, it is useful in cases where the functional material is mixed with the other materials, is added internally therein, or is used for coating applications. Coating it on the surface of a filter which is formed by corrugate processing a paper product, and the like, (as a honeycomb shape, for instance), and so forth, have been carried out.

The functional material can be applied in various modes, such as using it as a powder per se or by putting it in a bag or by sandwiching it between layers, pelletizing or molding the powder, producing molded products by adding the powder internally in polymer components or ceramic components, coating or impregnating arbitrary physical objects with a coating liquid prepared from the powder using a binder, if necessary, and attaching it additionally to nonwoven clothes.

Depending on the aforementioned applications, the particle diameter of the functional material is controlled to suitable sizes. For example, it is preferable to control the average particle diameter of the functional material to 20 μm or less, especially 15 μm or less. Regarding the lower limit, it is not limited in particular, however, it is possible to adapt it to around 1 μm, further, on an order of submicron (0.1 μm). Moreover, in cases where it is added internally in polymer components to produce fibers, it is desirable to be a particle diameter of 3 μm or less. Regarding functional materials described in the following embodiment modes, they can be utilized for similar applications, and it is desirable to employ similar modes.

Said catechins are catechin being derived from tea. As for the catechins used in the present invention, those whose importance is high are tea-derived-catechin drug products whose concentrations of catechins are heightened. The major components of tea-derived catechin are epigalocatechin, epigalocatechin gallate, epicatechin, epicatechin gallate, and the like. Since it is not needed necessarily to isolate it to the independent components, it is possible to suitably use the tea-derived-catechin drug products comprising mixtures of these as they are. As for commercially available tea-derived-catechin drug products, 30% products, 50% products, 60% products, 70% products, 80% products, 90% products, and the like, are available as products whose catechin purity is prescribed, and can be utilized in compliance with objects. In particular, the 90% products are such that the epigalocatechin gallate of high anti-oxidation power becomes the main component.

Said vitamins can be selected from vitamin, vitamin derivatives, and vitamin-like substances acting like vitamin.

Vitamin is one which operates importantly for the metabolism within human body in a trace amount, 13 types of compounds are referred to as vitamin currently. As examples, ascorbic acid, retinol, d-δ-tocopherol, pantothenic acid, nicotinic acid amide, biotin, phytonadione, and folic acid are named. In particular, ascorbic acid is preferable.

Further, for the purpose of improving the effectiveness or usability of vitamin, various derivatives have been developed. Major derivatives of vitamin are set forth below: ascorbil ethyl, ascorbil glucoside, (ascorbil/cholesteril) sodium phosphate, (ascorbil/tocopheril) potassium phosphate, ascorbil methyl silanol pectin, ascorbil phosphoric acid (Mg/K), ascorbil phosphoric acid (Mg/Na), ascorbil phosphoric acid (Mg/zinc), phosphoric acid ascorbil Ca, phosphoric ascorbil acid Na, phosphoric ascorbil acid Mg, ascorbil phosphoric acid 3Na, ascorbil aminopropyl phosphate, ascorbic acid Ca, ascorbic acid Mg, tetra-hexylydecyl ascorbilate, ascorbic acid polypeptide, ascorbic acid sulfate 2Na, ascorbil stearic phosphate, tetra-2-hexyldecanoic acid L-ascorbil, chitosan ascorbic acid, pantenil ethyl, pantothenic acid amid MEA, pantothenic acid polypeptide, dicarboethoxy pantothenic acid ethyl, phosphoric acid tocopherol 2Na, dicaprylylic acid pyridoxine, phosphoric acid pyridoxal, nicotinic acid hexyl, nicotinic acid tocopherol, nicotinic acid benzyl, nicotinic acid methyl, and the like. In particular, the derivatives of ascorbic acid, for example, ascorbil phosphoric acid Ca, ascorbil phosphoric acid Mg, and ascorbil phosphoric acid Na are preferable.

For the vitamin-like substances acting like vitamin, vitamin P is available, for example. The vitamin P is a generic name of hesperidin, rutin, and the like, and its actions, such as the strengthening of blood capillaries and the inhibition of blood-vessel permeability, have been known since a long time ago. However, since its deficiency diseases have not become apparent, it is excluded from vitamin.

As for the vitamins, ascorbic acid and the derivatives of ascorbic acid are especially preferable.

Said tannins can use commercially available tannic acid which is refined. Moreover, it is possible to use extracts of natural plants containing tannic acid or their semi-refined products, such as Gallae Rhois and gallic acid, as they are. It is possible to use pyrogallol, gallic acid and gallate ester as well. As for the tannins, tannic acid is especially preferable.

Said natural moisturizing factors are defined widely as natural components having moisturizing actions in the present invention, and major ones are exemplified below. It is possible to name hyaluronic acids comprising hyaluronic acid and its salts, amino acid, polyamino acid, amino-acid-based surfactants, pyrrolidone carboxylic acid and its salts, N-acetylglucosamine, animal and plant polysaccharide, co-enzyme Q10, rice powders, gelatin, oligosaccharide, monosaccharides, saponins, vegetable peptide, phospholipid, sericin, albumin, chondroitin, ceramide, collagen, and chitin as well as chitosan.

As for the hyaluronic acid salt, it is possible to name sodium hyaluronate, and the like.

As for the preferable amino acid, phenylalanine, glycin, proline, cystine, lycine, theanine, serine, asparaginic acid, valine, leucine, isoleucine, lysine, glutamine, arginine, ellagic acid, and the like, are available. As for the preferable polyamino acid, it is possible to name polylysine, polyglutamic acid, and so forth. As for the pyrrolidone carboxylic acid and its salts, it is possible to name pyrrolidone carboxylic acid, pyrrolidone sodium carboxylate, and so on. The N-acetylglucosamine is a type of sugar, and it is possible to name those which are made from chitin as a raw material.

As for the animal and plant polysaccharide, (1) it is possible to name cyamoposis gum, locust bean gum and queens seed gum which are seed polysaccharide, carrageen and alginic acid which are seaweed polysaccharide, Arabic gum and tragacanth gum which are resin polysaccharide, and the like, as the plant polysaccharide, and (2) as for the animal polysaccharide, collagen peptides, which are extracted from fish guts, fish scales, and so forth, are available. As for the oligosaccharide, it is possible to name xylobiose, trehalose, and so on. As for the monosaccharides, it is possible to name glucose, mannose, fructose, ribose, and the like.

The saponins are such that it is possible to name those being derived from plants which are included in tea, liquorice, Panacls Japonici Rhizoma, soybean, bupleurum root, Gynostemma pentaphylla, loofa, Polygae Radix, Japanese bellflower, senega, ophiopogonis Tuber, Akebiae Caulis, Anemarrhena rhizome, Achyranthis Radix, Smilax Glabra, and so forth. As for the vegetable peptide, it is possible to name hydrolyzed wheat powders, soy-bean protein hydrolyzed products, peptide of pea, and so on.

The phospholipid is a type of complex lipid, is lipid which has a phosphate group and the other atomic groups which usually include nitrogen, and is one which are included in egg yolk, natural butter, the embryos of wheat and corn, and soy bean relatively abundantly. The sericin is a type of protein which is included in silk thread. The albumin is a type of protein which are distributed in organisms widely, and it is possible to name egg albumin, blood-serum albumin, and the like, as for the animal one. As for the plant one, it is possible to name leucosin included in wheat, legumelin included in soy bean, and so forth. The chondroitin is such that it is possible to name those being derived from fishes, and plant-derived ones. The ceramid is such that it is possible to name animal-derived ones and plant-derived ones. The collagen is such that animal-derived ones and plant-derived ones are available and heated gelatin and enzymatically-decomposed collagen peptide can be utilized as well.

The chitin is polysaccharides which are included in the shells of crabs, shrimps, and the like, or the skins of insects, fungus, algaes, lower creatures, and so forth. The chitosan is one which is obtained by N-deacetylating the chitin.

As for the natural moisturizing factors, sodium hyaluronate, pyrrolidone carboxylic acid, cysteine, asparaginic acid, collagen peptide, and N-acetylglucosamine are especially preferable.

As the functional components, it is possible to include lactic acid bacteria, citric acid, and amino acid-based surfactants as well.

The essential oils derived from plants are plant-derived effective components which have at least one property selected from the group consisting of anti-microorganism properties, deodorizing properties, anti-allergenic properties, anti-oxidation properties, anti-inflammation properties, relaxation properties, aroma-therapeutic properties, moisturizing properties, noxious-minor-creature rejective actions, and which are exemplified below. For example, essential oil-related, crude drug-related, and the other related components can be exemplified. As for the essential oil-related ones, effective components, which are derived from Anise, West Indian Sandalwood, Angelica, Hibiscus abelmoschus, Immortelle, Ylang-Ylang, Inula, Winter green, Estragon, Elemi, Oregano, Orange, Chamomile Roman, Cajeput, Garlic, Cardamom, Galbanum, Camphor, Catnip, Caraway, Carrot seed, Guaiacwood, Cumin, Clary Sage, Clove, Grapefruit, Cade, Coriander, Cypress, Sandalwood, Santolina, Cederleaf, Cederwood, Citronella, Cinnamon, Jasmine, Juniper, Ginger, Star Anis, Spruce, Sage, Savory, Geranium, Celery, St.Jhon's Wort, Thyme, Taget, Tanacetum ammuum, Tarragon, Tangerine, Thuja, Tea Tree, Dill, Turpentime, Niauli, Nutmeg, Neroli, Violet, Pine, Basil, Parsley, Birch, Patchouli, Honeysuckle, Verbena, Pennyroyal, Rose, Palmarosa, Hyssop, Pimiento, Fir, Fennel, Petitgrain, Black pepper, Frankincense, Vetiver, Benzoin, Mint, Bergamot, Lime blossom, Marjoram, Myrtle, Mandarin, Melissa, Myrrh, Yarrow, Eucalyptus, Lime, Lavandin, Lavender, Litsea Cubeba, Lemon, Lemongrass, Rosewood, Rosemary, and Laurel, can be named. As for the crude drug-related ones, effective components, which are derived from Japanese laurel, Chinese parasol, Madder, Mallotus japponicus, Gambir, Aloe, Apricot, Epimedium grandiflorum, Polygonum cuspidatum, Taxus cuspidata, Fig, Achyranthes japonica, Turmeric, Cassia obtusifolia L., Japanese pagoda tree, Astragalus, Scutellaria root, Phellodendron bark, Coptidis Rhizoma, Plantain, Atractylodes japonica KOIDZUMI exKITAMURA., Panax ginseng C.A.MEYER, Hypericum villosa, Persimmon, Uncaria rhynchophylla MIQUEL, Ground ivy, Japanese valerian, Brassia juncea Cosson., Pinellia ternata BREITENBACH, Chinese quince, Artemisia capillaris THUNBERG, Licorice root, Trichosanthes kirillowii var. japonicum KITAMURA, Platycodon root, Catalpa fruit, Rumex japonicus, Phellodendron amurense RUPRECHT, Astragalus membranacens BUNGE, Agrimonia pilosa, Licium chinese MILLER, Sophora root, Kudzu, Gardenia, Spicebush, Mulberry, Shizonepeta spike, Cassia bark, Geranium herb, Oriental benzoar, Scutellaria baicalensis GEORGE, Evoidiae fruit, Bupleurum root, Alisma orientale JUZEPCZUK, Zizyphus vulgaris LAM. var. spinosus BUNGE, Smilax china, Cornus fruit, Japanese pepper, Rehmannia root, Lithospermum root, Beefsteak plant, Paeony root, Ophiopogon japonicus KER-GAWLER, Ginger, Nandina domestica, Japanese honeysuckle, Sappanwood, Field horsetail, Japanese parsley, Cnidium rhizome, Swertia herb, Rhubarb, Bitter orange, Aralia elata, Salvia militiorhhiza, Dandelion, Anemarrhena rhizome, Clove, Bitter orange peel, Sinomenium acutum, Polygonum multiflorum, Japanese Silverleaf, Japanese angelica root, “Dokudami,” Aconite, Nutmeg, Polygonatum falcatum, Nandina, Picrasma quassioides, Elderberry, Garlic, Rosa multiflora THUNB., “Hakusenbi,” Mentha herb, Tear grass, Anemarrhena asphode loides BUNGE, Alpinia japonica MIQ, Cassia torosa CAVANILLES, Cyperus rotundus L., Glehnia root, Plectranthus japonicus, Angelica dahurica root, Convolvulus, Loquat, Betelpalm, Hoelen, Thoroughwort, Safflower, Peony, Ephedra herba, Silvervine, Bupleurum falcatum L., Althaea, Leonurus sibiricus, Peach, Dioscorea japonica THUNB., Saxifrage, Mugwort, Gentian, Forsythia fruit, and “Reishi,” can be named. As for the other related ones, effective components, which are derived from Ginkgo leaves, Storax, Kaffir-lime, Artemisia capillaris THUNBERG, Citrus seeds, Guava tea, “Kumazasa” bamboo, Coffee beans, Stevia, Soybean, Bamboo, Knotiveed, Checker tree, Galanga, Leeks, Pimenta dioica MERRILL, Japanese cypress, Japanese cypress thiol, Phytoncide, Grapes' fruit skins, pepper, Japanese white bark magnolia, “Hokkoshi,” “Mosochiku” bamboo, Rice hull, “Yamabushitake” bamboo, and Horse radish, can be named. Moreover, as for the essential oils being derived from plants, greenery alcohol (CH₃CH₂CH═CHCH₂CH₂OH), and greenery aldehyde (CH₃CH₂CH═CHCH₂CHO) are included.

The functional ingredient is a fine particle. Since a fine particle is such that the superficial area is large, it is likely to be loaded with loading substances. The functional ingredient can be selected from the group consisting of inorganic ingredients comprising colloidal silica, calcium silicate, ethyl silicate, sodium silicate, potassium silicate and lithium silicate, which are silic acid-based ones, or calcium aluminate, β-alumina, boehmite and alumina sol, which are alumina-based ones, or calcium phosphate, aluminum phosphate and magnesium phosphate, which are phosphoric acid-based ones, and organic ingredients comprising cellulose, cellulose acetate, carboxymethylcellulose and polyvinyl alcohol. As for the functional ingredient, colloidal silica, cellulose acetate, cellulose, and calcium phosphate are especially preferable.

In the present invention, a fine-particulate mode is used for the functional ingredient and functional material, however, it is noted additionally that there are possibilities of demonstrating similar effects in modes other than the fine-particulate one, such as thin-film modes and molded bodies, as well.

(Functional Material #2 of First Embodiment Mode)

A further functional material of the present invention is characterized in that it is producible by a production process having a dropletizing step, and a drying step. The dropletizing step is a step in which an aqueous slurry, which has two or more loading components selected from functional components comprising the group consisting of catechins, vitamins, tannins, natural moisturizing factors and essential oils being derived from plants, and a functional ingredient, which is a fine particle, is turned into a fine droplet state. The drying step is a step in which the fine droplet is dried by contacting it with a hot air.

Since the functional components and functional ingredient are such that it is possible to apply those which are similar to the ones explained in the above-described First Embodiment Mode, and, regarding the dropletizing step and drying step, since they are similar to steps, which are described later in the production process, further explanations are omitted herein.

(Production Process of Functional Material #3 of First Embodiment Mode)

A production process of the present invention has it has: a dropletizing step of turning an aqueous slurry having two or more loading components selected from functional components comprising the group consisting of catechins, vitamins, tannins, natural moisturizing factors and essential oils being derived from plants, and a functional ingredient being a fine particle into a fine droplet state; and a drying step of drying the fine droplet by contacting it with a hot air. The dropletizing step and drying step can be carried out in a bath of an appropriate size.

Regarding the functional components and functional ingredient, since it is possible to apply those which are similar to the ones explained in the above-described First Embodiment Mode, further explanations are omitted herein.

A solvent of the aqueous slurry is usually water, however, it does not matter if it includes a proper amount of organic solvent (alcohol, and the like).

A solid-content concentration of the aqueous solution is not limited in particular, however, it can usually be 1% by mass or more and 70% by mass or less, especially 3% mass or more and 60% by mass or less, above all 5% by mass more or more and 50% by mass or less, in view of finely-pulverizing and thermal energy.

The diameters of the finely-pulverized droplets are usually classified “spray” when they exceed 10 μm, and “mist” when they are 10 μm or less. It is especially preferable that they can be the latter, “mist.”

The aforementioned dropletizing is done using a rotary disk, a pressurizing nozzle, a binary-fluid nozzle, a quaternary-fluid nozzle, and the like. Especially, since the quaternary-fluid nozzle can turn droplets into “mist” and spray it abundantly, it is preferable.

In the quaternary-fluid nozzle, a knife-shaped nozzle edge is provided with two channels of gas passage and fluid passage, respectively, or 4 channels in total. The nozzle-edge leading end is constructed so that a collision focus, at which a liquid (aqueous slurry), having flowed on inclined surfaces being the two-channel liquid flowing surfaces, gathers to one point. The nozzle edge can desirably be provided with a linear portion of an appropriate length depending on spraying amounts. The high-speed gaseous fluid coming out of the gas slit extends the liquid, which comes out of the liquid slit like springing out therefrom, thinly, while mixing it by the liquid flowing surfaces. The extended liquid, which flows out of the two channels, collides at the collision focus in the edge leading end, is pulverized more finely by the shock waves generated thereat, and becomes droplets of a few μm.

The quaternary-fluid nozzle is an advantageous method from the following viewpoints: the fine droplet diameters whose average particle diameter is a few μm are obtainable by the aforementioned method; it becomes droplets with uniform grain size; it is possible to arbitrarily control the droplet diameters by vapor-liquid ratio; and it is possible to spray abundantly with a single nozzle.

In the drying step, the dropletized functional material is dried by contacting it with a hot air to load the functional components on the functional ingredient. Here, the temperature control is especially important. Depending on the types of, the respective loading components, a temperature set-up, at which the components neither degenerate nor evaporate off, and at which the functional components can be loaded on the functional ingredient fully.

As for the drying step, not limited to “up down falling types” in which the droplets fall from top to down, it is possible to use various ones, such as “blowing-up types,” “horizontal types,” and “cyclone types.”

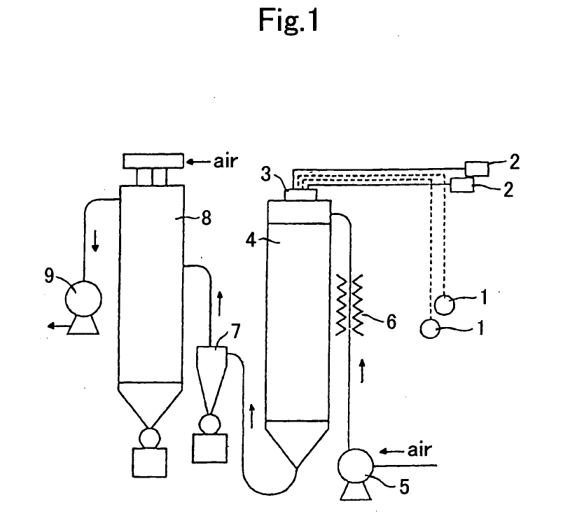

A specific apparatus for carrying out the production process is exemplified in FIG. 1. The present apparatus is a cyclone type apparatus which has liquid supplying means 1, gas supplying means 2, a nozzle (quaternary-fluid) 3, an apparatus body (bath) 4, an air blower 5, a heater 6, a cyclone 7, a bag filter 8, and an air discharger 9.

The liquid supplying means 1 and the gas supplying means 2 are such that there are 2 channels, respectively, and they are channels for supplying the aqueous slurry and the gas to the quaternary-fluid nozzle 3, respectively. The quaternary-fluid nozzle 3 is disposed at the top of the bath 4. The quaternary-fluid nozzle 3 is equipped with two liquid supplying channels (not shown) and two gas supplying channels (not shown). The liquid supplying channels and gas supplying channels are paired, and are disposed symmetrically. To the liquid supplying channels, the liquid supplying means 1 for supplying the aqueous slurry is connected, and, to the gas supplying channels, the gas supplying means 2 for supplying air is connected. The quaternary-fluid nozzle 3 is equipped with the above-described bottom nozzle-edge portion (not shown) having a leading end at the symmetrically central portion in which the liquid supplying channels and gas supplying channels are disposed, and the gas supplying channels are disposed so as to flow the spouting gas along the surface of the bottom nozzle-edge portion and collide the gas at the leading end of the bottom nozzle-edge portion. The liquid supplying channels are means for supplying the aqueous slurry at the midway of the gas flow from the gas supplying channels to spray it accompanied by the gas flow.

The bath 4 is a cylinder-shaped member whose inside is hollowed. At the top, the quaternary-fluid nozzle 3 is disposed, and additionally an air-blower opening, to which the air is delivered from the air blower 5, opens. The air-blower opening is disposed in a direction in which the flow of the air introduced into the bath 4 descends while rotating along the inner surface. Between the air blower 5 and the air-blower opening, the heater 6, which can heat the air delivered into the bath 4 to a predetermined temperature, is disposed. At the bottom of the bath 4, there is a cone-shaped portion in which the apex is present on the bottom side, and it is equipped with a discharge opening which can discharge the contents within the bath 4 through the apex of the cone-shaped portion.

The discharge opening of the bath 4 is connected to the cyclone 7. The discharge opening of the cyclone 7 is connected to the bag filter 8. To the bag filer 8, the air discharger 9 is connected.

The aqueous slurry is supplied to the liquid supplying channels of the quaternary-fluid nozzle 3 through the liquid supplying channels 1. Simultaneously, the gas is supplied to the gas supplying channels of the quaternary-fluid nozzle 3 from the gas supplying channels 2. As a result, the aqueous slurry is turned into a mist, and is sprayed into the bath 4 (dropletizing step). The air delivered from the air blower 5 is heated by the heater 6, and is introduced into the bath 4. Within the bath 4, the mist (fine droplets) of the aqueous slurry contacts with the heated air (hot air) so that the mist is dried, and thereby the functional material is produced (drying step). By drying it with the hot air, the functional components contained as the loading components are loaded on the functional ingredient.

The produced functional material falls down the air blower 5 within the bath 4, together with the hot air from the air blower 5, and is sent to the cyclone 7 from the bottom cone. Within the cyclone 7, the most part of the functional material is collected. The functional material, which has not been collected at the cyclone 7, is sent out to and collected at the bag filter 8. At the bag filter 8, a negative pressure is generated by the air discharger 9, and the outside air is sucked in, in addition to the hot air accompanying the functional material which is introduced from the cyclone 7. Note that the recovery (capture) of the functional material can be carried out using both of the cyclone 7 and bag filer 8, or can be carried out using either one of them.

The mixing ratio of two arbitrary components among said functional components which the loading components contain is 1 to 100 or more and 100 to 1 or less on mass basis, it is possible to design the rate variously depending the types and conditions of the functional ingredient on which they are loaded, and in compliance with objects.

The proportion (as solid contents) of the loading components to the functional ingredient can be set up variously, however, it is desirable that the loading components can be 1 part by mass or more and 70 parts by mass or less, especially 3 parts by mass or more and 60 parts by mass or less, above all 5 parts by mass or more and 50 parts by mass or less, when the functional ingredient is taken as 100 parts by mass. Being within this range, the functionalities are demonstrated sufficiently, and the functionalities can be sustained sufficiently.

The set-up temperature of the hot air can preferably be set up so that the inlet temperature can be 100° C. or more and 300° C. or less (especially 100° C. or more and 250° C. or less). Regarding the exhaust temperature, it can be set up at 65° C. or more and 250° C. or less (especially 65° C. or more and 150° C. or less), and at the same time can preferably be set up at a lower temperature than the inlet temperature by 30° C. or more (especially 50° C. or more). Being the lower limits or more of these ranges, the drying time is appropriate so that the loading of the functional components on the surface of the functional ingredient or to the inside thereof can be done sufficiently. Even when the functional material is used in such a manner that it is brought into contact with water, the sustainability of releasability continues. Moreover, being the upper limits or less, the loading components do not degenerate so that there is no fear that they have evaporated off. The aforementioned temperature ranges are temperature conditions under which the objective product can be obtained efficiently.

The average particle diameter of the functional material exhausted from within the bath can preferably be controlled to 20 μm or less, especially 15 μm or less. The control of the average particle diameter can be achieved by controlling the size of droplets, the particle diameter of the functional-ingredient component in the aqueous slurry, and the like. Regarding the lower limit, it is not limited in particular, however, it is possible to adapt it to around 1 μm, further, on an order of submicron (0.1 μm). Moreover, when silica particulates of arbitrary size, and so forth, are added, since the functional material might be pulverized more finely than the size being controlled by the size of droplets when drying the droplets, it can be utilized in the particle-diameter control of fine powder.

The silica particulates, and the like, can preferably be adapted to particulates of an average particle diameter of 1.5 μm or less, for the purpose of finely pulverizing the functional material to be produced.

(Functional Material #1 of Second Embodiment Mode)

The functional material of the present embodiment mode is silky particulates and the touch is smooth as well, because the mode, in which the functional ingredient is adhered onto the surface of ceramic particles or covers it, becomes the major component. The functional component is loaded on the superficial functional ingredient.

The application of the functional material is useful for a variety of applications beginning with cosmetic materials, food materials, health food materials, filter materials for air conditioners, air cleaners, cleaners, and the like, clothes, bedclothes-related materials, hygienic materials, footwear materials, carpet materials, kitchen utensils, toiletry utensils, interior materials for buildings or vehicles, building materials, medical materials, agricultural or gardening materials, and packaging materials.

The functional material can be applied in various modes, such as using it as a powder per se or by putting it in a bag or by sandwiching it between layers, pelletizing or molding the powder, producing molded products by adding the powder internally in polymer components or ceramic components, coating or impregnating arbitrary physical objects with a coating liquid prepared from the powder using a binder, if necessary, and attaching it additionally to nonwoven clothes.

Depending on the aforementioned applications, the particle diameter of the functional material is controlled to suitable sizes. For example, it is preferable to control the average particle diameter of the functional material to 20 μm or less, especially 15 μm or less. Regarding the lower limit, it is not limited in particular, however, it is possible to adapt it to around 1 μm, further, on an order of submicron (0.1 μm). Moreover, in cases where it is added internally in polymer components to produce fibers, it is desirable to be a particle diameter of 3 μm or less.

Since the functional material of the present embodiment mode can be produced at a lower temperature than conventionally, it is more likely to demonstrate the functions than conventionally in applications using natural-product-derived functional components which are likely to decompose or evaporate off by heat.

Moreover, by changing the compounding ratio of the ceramic particles with the functional ingredient and functional component, it is possible to alter the amounts of the functional ingredient and functional component, which adhere on or cover the surfaces of the ceramic particles. Thus, it is possible to arbitrarily design the releasability of the functional component contained in or loaded on the functional ingredient, in compliance with objects. For example, when the amount of the ceramic particles is reduced, the amount of the adhering or covering functional ingredient and functional component per a superficial area of the ceramic particles becomes great so that those which emit the functional component by a small amount and have longer lives can be obtained. Moreover, when the amount of the ceramic particles is made greater, the amount of the adhering or covering functional ingredient and functional component per a superficial area of the ceramic particles decreases so that those which emit it abundantly at once and have shorter lives can be obtained.

Since the functional component in the functional material of the present embodiment mode is the same as those explained in the above-described First Embodiment Mode, further explanations are omitted herein. Moreover, the method of loading the functional component on the functional ingredient is also the same as those explained in the First Embodiment Mode, except that those in which the functional component is a single species are included as well in the present embodiment mode, on the contrary, adding two or more of the functional components is essential in the First Embodiment Mode.

The functional ingredient is fine particles in dry state. Since fine particles are such that superficial areas are large, they are likely to load functional substances thereon. The functional ingredient is selected from the group consisting of cellulose, cellulose derivatives, and polyvinyl alcohol. As for the cellulose derivatives, cellulose acetate and carboxymethylcellulose can be named. As for the functional ingredient, cellulose and cellulose acetate are especially preferable.

As for the ceramic particles, various clay minerals, oxides, hydroxides, composite oxides, nitrides, carbides, silicides, borides, zeolite, cristobalite, diatomaceous earth, and polyvalent metallic salts of silic acid, or calcium carbonate can be named. As for the clay minerals, sepiolite, cordierite, kaoline, bentonite, and the like, can be named. As for the oxides, alumina, titania, silica, zirconia, magnesia, zinc oxide, and so forth, can be named. As for the hydroxides, the hydroxides of aluminum, zinc, magnesium calcium and manganese, and so on, can be named. An example of the composite oxide is alum. Examples of the nitrides are silicon nitride, boron nitride, and the like. Examples of the carbides are silicon carbide, boron carbide, and so forth. As for the polyvalent metallic salts of silic acid, aluminum salts, zinc salts, magnesium salts, calcium salts, manganese salts, and so on, can be named. As for alkali metal salts of silic acid, lithium salts, sodium salts, potassium salts, and the like, can be named. Moreover, inorganic photocatalysts, such as photocatalytic titanium oxide, can be named as an example. As for the ceramic particles, silica fine particles and sepiolite are desirable.

In the present invention, a fine-particulate mode is used for the functional ingredient and functional material, however, it is noted additionally that there are possibilities of demonstrating similar effects in modes other than the fine-particulate one, such as thin-film modes and molded bodies, as well.

(Functional Material #2 of Second Embodiment Mode)

A further functional material of the present invention is characterized in that it is producible by a production process having a dropletizing step and a drying step. The dropletizing step is a step in which an aqueous slurry having: ceramic particles; a functional ingredient selected from the group consisting of cellulose, cellulose derivatives and polyvinyl alcohol; and a functional component selected from the group consisting of catechins, vitamins, tannins, natural moisturizing factors and essential oils being derived from plants is turned into a fine droplet state. The drying step is a step in which the fine droplet is dried by contacting it with a hot air.

Since the functional component and functional ingredient are such that it is possible to apply those which are similar to the ones explained in above-described #1 of Second Embodiment Mode, and, regarding the dropletizing step and drying step, since they are similar to steps, which are described later in the production process, further explanations are omitted herein.

(Production Process of Functional Material #3 of Second Embodiment Mode)

A production process of the present invention has: a dropletizing step of turning an aqueous slurry into a fine droplet state, the aqueous slurry having: ceramic particles; said functional ingredient selected from the group consisting of cellulose, cellulose derivatives and polyvinyl alcohol; and a functional component selected from the group consisting of catechins, vitamins, tannins, natural moisturizing factors and essential oils being derived from plants; and a drying step of drying the fine droplet by contacting it with a hot air. The dropletizing step and drying step can be carried out in a bath of an appropriate size.

Regarding the functional component and functional ingredient, since it is possible to apply those which are similar to the ones explained in above-described #1 of Second Embodiment Mode, further explanations are omitted herein. Moreover, as for specific examples of the production process and using apparatus therefor, since they are similar to those explained in #3 of First Embodiment Mode, further explanations are omitted.

The proportion (as solid contents) of the loading component and functional ingredient to the ceramic particles can be set up variously, however, it is desirable that the functional component can be 1 part by mass or more and 70 parts by mass or less, especially 3 parts by mass or more and 60 parts by mass or less, above all 5 parts by mass or more and 50 parts by mass or less, when the ceramic particles are taken as 100 parts by mass. Being within this range, the functionality is demonstrated sufficiently, and the functionality can be sustained sufficiently.

Moreover, when the ceramic particles are taken as 100 parts by mass, it is desirable that the functional ingredient can be 10 parts by mass or less. When the ceramic particles are less, the amount of the adhering or covering functional ingredient and functional component per a superficial area of the ceramic particles becomes great so that those which emit the functional component by a small amount and have longer lives can be obtained. Moreover, when the amount of the ceramic particles is made greater, the amount of the adhering or covering functional ingredient and functional component per a superficial area of the ceramic particles decreases so that those which can emit it abundantly at once and have shorter lives can be obtained. That is, by changing the compounding ratio of the ceramic particles with the functional ingredient and functional component, it is possible to alter the amounts of the functional ingredient and functional component, which adhere on or cover the ceramic particles. Thus, it is possible to arbitrarily design the releasability of the functional component contained in or loaded on the functional ingredient, in compliance with objects.

The set-up temperature of the hot air can preferably be set up so that the inlet temperature can be 100° C. or more and 300° C. or less (especially 100° C. or more and 250° C. or less). Regarding the exhaust temperature, it can be set up at 65° C. or more and 250° C. or less (especially 65° C. or more and 150° C. or less), and at the same time can preferably be set up at a lower temperature than the inlet temperature by 30° C. or more (especially 50° C. or more). Being the lower limits or more of these ranges, the drying time is appropriate so that the loading of the functional component on the surface of the functional ingredient or to the inside thereof can be done sufficiently. Even when the functional material is used in such a manner that it is brought into contact with water, the sustainability of releasability continues. Moreover, being the upper values or less, the loading component does not degenerate so that there is no fear that it has evaporated off. The aforementioned temperature ranges are temperature conditions under which the objective product can be obtained efficiently.

The average particle diameter of the functional material exhausted from within the bath can preferably be controlled to 20 μm or less, especially 15 μm or less. The control of the average particle diameter can be achieved by controlling the size of droplets, the particle diameters of the ceramic particles and functional ingredient, and the like. Regarding the lower limit, it is not limited in particular, however, it is possible to adapt it to around 1 μm, further, on an order of submicron (0.1 μm). Moreover, due to the presence of the ceramic particles, since the functional material might be pulverized more finely than the size being controlled by the size of droplets when drying the droplets, it can be utilized in the particle-diameter control of fine powder.

The ceramic particles can preferably be adapted to particulates of an average particle diameter of 1.5 μm or less, for the purpose of finely pulverizing the functional material to be produced.

[#1 of Third Embodiment Mode: Functional Member: Filter]

The functional member of the present embodiment mode is a member which can emit a functional component into atmospheres in which it is used. In particular, it can be applied to filters used in air conditioners (air-conditioning machines), air-cleaning machines, cleaners, refrigerators, “futon”-drying machines, humidifiers, dehumidifiers, hair driers and ventilating fans, and apparatuses which move air (in which air moves), such as electric fans, fans, foldable fans, ventilating openings, screen doors and “sudare” blinds.

The functional member of the present invention has two or more types of loading components, and a support.

The loading components are selected from functional components comprising the group consisting of catechins, vitamins, natural moisturizing factors and tannins. The present functional member can be employed for filters for air transmission, filters which can be used in air conditioners, air cleaners, facial-treatment devices. As for the modes of the functional member (filter), they are not limited in particular, however it is possible to exemplify modes such as porous bodies, honeycomb bodies and thin-film-shaped members which are formed as bellows shapes or cylinder shapes.

The functional member is a member through which air can transmit in one direction at least. The loaded loading components are emitted, and the like, with respect to air which passes through the inside, and thereby the functional components operate. When it includes ascorbic acid as a functional component, it is preferable to adjust the pH of the functional member on an acidic side (pH 5 or less is preferable, and 3 or less is more preferable). The pH control can be carried out by selecting a support which is acidic by itself, and using a pH-adjusting agent (being possible by mixing it in the production of a support, or mixing it with a loading component to load it on a support). In addition, depending on the types of the functional components, it is preferable to properly control the pH of the functional member, the water content, and the like.

(Loading Components)

Since the loading components can employ the components, which have been explained in the above-described First Embodiment Mode and Second Embodiment Mode, as they are, further explanations are omitted.

(Support)

The support is a member in which the loading components are contained in or loaded on the surface or in the inside thereof. The support is a member which has a hole which is continuous from one of the surfaces to the other one of the surfaces so as to enable air to pass through in one direction at least. For example, porous bodies which have continuous cells, or fiber aggregates, and the like, can be named. The sizes of pores formed in porous bodies can be selected from a μm order to a mm order, a cm order, and the like, depending on fields to which it is applied. As for the porous bodies, the aggregates of granulated bodies, those made by solidifying granulated bodies, honeycomb-shaped ones, and so forth, can be exemplified. Honeycomb-shaped support can be produced by ordinary methods, such as extrusion molding. Moreover, as for the fiber aggregates, ordinary cloths, nonwoven cloths, compression-molded bodies of fibers, and the like, can be named.

The loading of the loading components to the support can be carried out by loading them after producing the support, or can be carried out by molding the shape of the support in such a state that the loading components are added to a precursor substance for shaping the support, and the like. For example, it is possible to load them by immersing-drying the molded support into a liquid in which the loading components are suspended or dissolved in a proper solvent. Moreover, it can be carried out as well by mixing the loading components with a material, which is prior to extrusion molding a honeycomb-shaped substance. Moreover, in order to load them on a fiber aggregate, it is possible to have the loading components contained in fibers constituting the fiber aggregate. In order to have the loading components contained in fibers, a method of kneading the loading components in advance in a fiber spinning step, or a method of immersing spun fibers into a solution of the loading components, and so forth, can be named.

The support can be constituted of ceramic, polymer materials, and the like. For example, it can be constituted of silicates, oxides, such as alumina, ceria, zirconia, titania and silica, and composite oxides of these, and ceramic, such as clay minerals; synthesized polymer materials, such as polyester, polyamide, acrylic resin and polyolefine, organic polymer materials, such as (semi) natural polymer materials like cellulose (paper, cotton, and the like). As for the ceramic, cordierite, sepiolite, and so forth, can be exemplified, and it can preferably be a porous material by itself microscopically.

(Other Constituent Elements)

The present functional member can contain necessary members in addition to the support and loading components. For example, there are additives used on the occasion of processing, such as dispersants and binders, which become necessary for using ceramic powders in extrusion molding, and the like. For example, carboxymethylcellulose can be exemplified.

Moreover, for the purpose of loading the loading components on or containing them in the support more stably, it is possible to add particulate-shaped functional ingredient. Since the functional ingredient is particulates, the superficial area is large so that it is likely to interact with the loading substances. In this instance, the loading components can preferably be bonded to the functional ingredient chemically. The functional ingredient is loaded on or contained in the support.

The functional ingredient is selected from the group consisting of inorganic ingredients comprising colloidal silica, calcium silicate, ethyl silicate, sodium silicate, potassium silicate and lithium silicate, which are silic acid-based ones, calcium aluminate, β-alumina, boehmite and alumina sol, which are alumina-based ones, calcium phosphate, aluminum phosphate and magnesium phosphate, which are phosphoric acid-based ones, and organic ingredients comprising cellulose, cellulose acetate, carboxymethylcellulose and polyvinyl alcohol. As for the functional ingredient, colloidal silica, cellulose acetate, cellulose and calcium silicate are especially preferable. Moreover, as the functional ingredient, two or more types of the materials can be mixed to use.

As for the pH-adjusting agent being capable of adjusting the pH of the functional member, it is possible to exemplify acids, such as inorganic acids like phosphoric acid, boric acid, sulfuric acid, hydrochloric acid and nitric acid, organic acids like acetic acid, formic acid, glycine, citric acid, succinic acid, phthalic acid and benzoic acid, and alkalis, such as ammonia, alkylamine, sodium hydroxide and potassium hydroxide, and the like. It is needless to say that the pH-adjusting agent can be materials which can demonstrate the pH-adjusting function as well as the other operations (an operation as a binder, an operation as the functional ingredient, and so forth) simultaneously.

[#2 of Third Embodiment Mode: Environment Modifying Apparatus]

The present environment modifying apparatus has a filter, air-sending-out means, and moisturizing means. The filter is such that it is possible to apply the functional member of the above-described embodiment mode as it is. The air-sending-out means is means for passing air through the filter. As for the air-sending-out means, fans, and the like, which are driven by electric motors, and so forth, can be exemplified. Places where the air-sending-out means is disposed do not matter whether they are on an upstream side or a downstream side with respect to the filter. That is, the air-sending-out means can be adapted to means for sending out air to the filter by disposing it on an upstream side with respect to the filter, or can be adapted to means for sending air from the filter by disposing it on an upstream side with respect to the filter. The moisturizing means is means for moisturizing the air passing the filter.

The loading component loaded, or the like, on the filter is such that the amount to be emitted varies depending on the presence of water content (the humidity of passing air). Accompanied by the water content included in air passing through the filter, the loading component is emitted. The water content included in air can be included not only as vapor but also as mist. The details will be described in examples, and, according to graphs for examining the relationship between the elution amount of ascorbic acid and the humidity in sent-out air, graphs which were obtained in experiments regarding ascorbic acid, the sigmoid curve is shown, in which the emission amount of ascorbic acid varies drastically at 70% humidity approximately. Namely, when the humidity of air passing through the filter is raised, the emission amount of ascorbic acid (functional component) rises.

(Functional Material #1 of Fourth Embodiment Mode)

A functional material of the present embodiment mode is one in which a functional component is loaded on a functional ingredient being a fine particle in dry state and being selected from organic polymer materials, and is a material which exhibits dispersibility with respect to linseed oil. By being provided with dispersibility with respect to linseed oil, it can be used suitably as fragrant cosmetics. Here, “exhibiting dispersibility with respect to linseed oil” is as described above. Further, by employing arrangements which do not contain inorganic components substantially, the touch can be improved.

As for the organic polymer material employable for the functional ingredient, polymer materials which are derived from animals and plants (that is, which are derived from nature), such as cellulose, rubbery substances and dietary fibers; polymer materials (derivatives) in which chemical modification is performed to these nature-derived polymer materials; and polyvinyl alcohol being a synthetic polymer, can be named. These materials can be divided roughly into being water soluble and being insoluble.

As for the insoluble polymer materials, cellulose, hemicellulose, lignin, pectin, Japanese gelatin, and chitin (as well as chitosan being its derivative) are available, for example. These polymer materials are appreciable in foods, and cellulose is derivable from vegetables, grains (bran, and the like, being the same hereinafter), beans, and so forth; hemicellulose is derivable from grains, beans, and soon; lignin is derivable from cocoa, grains, beans, and the like; pectin is derivable from unmature fruits and vegetables (being present on cell walls while being unmature, and becoming insoluble); the Japanese gelatin is derivable from agar-agar; and the chitin is derivable from the shells of crustacea, such as shrimps-crabs, locust, and so forth.

As for the water-soluble polymer materials, pectin, glucomannan, galactomannan, alginic acid, Carrageena, Guar Gum, and the like, are available, for example. These polymer materials are also appreciable in foods, and the pectin derives from fruits and vegetables; the glucomannan derives from alimentary yam paste, yam, and so forth; the galactomannan drives from oat, beans, and so on; the alginic acid derives from “Konbu” seaweed, “Wakame” seaweed, and the like; the Carrageenan derives from red algae, and so forth; the Guar Gum derives from the secretions of gua bean; and the Fucoidan derives from seaweeds.

Moreover, hydroxyethyl cellulose, hydroxypropyl cellulose, carboxylmethyl cellulose, cellulose acetate, and the like, which are the derivatives of cellulose, can be exemplified. Moreover, it is possible to employ materials in which a functional group (a diethylaminoethyl group (DEAE), an amino group, or an alkyl group) is introduced into cellulose to control the superficial affinity.

Moreover, by changing the compounding ratio of the functional ingredient and functional component, it is possible to alter the amount of the functional component, which adhere on or cover the surface of the functional ingredient. Thus, it is possible to arbitrarily design the releasability of the functional component contained in or loaded on the functional ingredient, in compliance with objects.

Since the functional component in the functional material of the present embodiment mode is the same as those explained in the above-described First Embodiment Mode, further explanations are omitted herein. Moreover, the method of loading the functional component on the functional ingredient is also the same as those explained in the First Embodiment Mode, except that those in which the functional component is a single species are included as well in the present embodiment mode, on the contrary, adding two or more of the functional components is essential in the First Embodiment Mode.

(Functional Material #2 of Fourth Embodiment Mode)

A further functional material of the present invention is characterized in that it is producible by a production process having a dropletizing step and a drying step. The dropletizing step is a step of turning an aqueous slurry having the functional ingredient and functional component, which are explained in #1 of Fourth Embodiment Mode, into a fine droplet state. The drying step is a step in which the fine droplet is dried by contacting it with a hot air.

Since the functional component and functional ingredient are such that it is possible to apply those which are similar to the ones explained in above-described #1 of Fourth Embodiment Mode, and, regarding the dropletizing step and drying step, since they are similar to steps, which are described later in the production process, further explanations are omitted herein.

(Production Process of Functional Material #3 of Fourth Embodiment Mode)

A production process of the present invention has a dropletizing step of turning an aqueous slurry having the functional ingredient and functional component, which are explained in #1 of Fourth Embodiment Mode, into a fine droplet state, and a drying step of drying the fine droplet by contacting it with a hot air. The dropletizing step and drying step can be carried out in a bath of an appropriate size.

Regarding the functional component and functional ingredient, since it is possible to apply those which are similar to the ones explained in above-described #1 of Fourth Embodiment Mode, further explanations are omitted herein. Moreover, as for specific examples of the production process and using apparatus therefor, since they are similar to those explained in #3 of First Embodiment Mode, further explanations are omitted.

The set-up temperature of the hot air can preferably be set up so that the inlet temperature can be 100° C. or more and 300° C. or less (especially 100° C. or more and 250° C. or less). Regarding the exhaust temperature, it can be set up at 65° C. or more and 250° C. or less (especially 65° C. or more and 150° C. or less), and at the same time can preferably be set up at a lower temperature than the inlet temperature by 30° C. or more (especially 50° C. or more). Being the lower limits or more of these ranges, the drying time is appropriate so that the loading of the functional component on the surface of the functional ingredient or to the inside thereof can be done sufficiently. Even when the functional material is used in such a manner that it is brought into contact with water, the sustainability of releasability continues. Moreover, being the upper limits or less, the loading component does not degenerate so that there is no fear that it has evaporated off. The aforementioned temperature ranges are temperature conditions under which the objective product can be obtained efficiently.

The average particle diameter of the functional material exhausted from within the bath can preferably be controlled to 20 μm or less, especially 15 μm or less. The control of the average particle diameter can be achieved by controlling the size of droplets, the particle diameter of the functional ingredient, and the like. Regarding the lower limit, it is not limited in particular, however, it is possible to adapt it to around 1 μm, further, on an order of submicron (0.1 μm).

### EXAMPLES

Next, the present invention will be further described while naming examples, however, the present invention is not one which is limited by these.

(Test #1)

Two types of the loading components were selected from the functional components, and were labeled functional components A and B. The loading component A was dissolved completely in water, the loading component B was added thereto, and was stirred until solids disappeared. To this, silica fine particles whose average particle diameter was 1.5 μm (“ZEOSEAL® 1100V” produced by TAKI KAGAKU Co., Ltd.) was added, and was stirred until solids disappeared, and subsequently a solution, in which a colloidal silica-40% water suspension (“ADELITE® AT-40” produced by ASAHI DENKA KOGYOU Co., Ltd.) or cellulose acetate (“CELL FLOW® TA-25” produced by CHISSO Co., Ltd.) was dispersed in a trace amount of ethanol, was added thereto and stirred, thereby preparing an aqueous slurry.

In Table 1, there are set forth the mixing ratios of the respective loading components and the hot-air set-up temperatures., Moreover, regarding the loading components and functional ingredients, they are set forth in Table 2. As for the loading components, tea-derived catechin was selected from the catechins to use; ascorbic acid and ascorbil magnesium phosphate were selected from the vitamins to use; tannic acid was selected from the tannins to use; and sodium hyaluronate, collagen peptide, pyrrolidone carboxylic acid, cysteine (amino acid) and N-acetylglucosamine were selected from the natural moisturizing factors to use.

The colloidal silica-40% water suspension was prepared so as to meet the respective functional components and so as not to gelate. The silica fine particles were added for the purpose of pulverizing the functional materials further finely. Within the obtained the functional materials as well, they could be utilized effectively for the purpose of controlling the quick activeness or slow activeness of the functional components and prolonging the lives of the functional materials.

As for an apparatus corresponding to FIG. 1, a spray-drying apparatus for research, “MICROMIST DRYER MDL-050-Type M” (one which was equipped with the quaternary-fluid nozzle) made by FUJISAKI DENKI Co., Ltd. was utilized, and the dropletizing step and drying step were carried out with respect to the aqueous slurries, and thereby producing-collecting the functional materials.

As for the functional ingredients, they were selected from collodial silica, being the inorganic ingredients, and cellulose acetate, being the organic ingredients.

Those which had two types of the loading components were labeled Example Nos. 1 through 10, those which had one type only were labeled Comparative Example Nos. 1 through 9, and they were adapted to test samples, respectively. Those with annexed numbers attached for both examples and comparative examples are such that annexed number 1 designates being combined with colloidal silica and annexed number 2 designates being combined with cellulose acetate. Moreover, samples which had the functional components alone, which were not loaded on the functional ingredients, are set forth in Table 3 as Comparative Example Nos. 10 through 11.

TABLE 1

| | | | Colloidal | | | | | |
|---|---|---|---|---|---|---|---|---|
| | | | Silica-40% | | | | | |
| | Loading | Loading | Water | Cellulose | Silica Fine | | | |
| | Component A | Component B | Suspension | Acetate | Particles | | Hot-air | Hot-air |
| | (Parts by | (Parts by | (Parts by | (Parts by | (Parts by | Water (Parts | Inlet Set-up | Outlet Set- |
| | Mass) | Mass) | Mass) | Mass) | Mass) | by Mass) | Temp. (° C.) | up Temp. (° C.) |
| Ex. No. 1 | 125 | 50 | 125 | 0 | 500 | 500 | 180 | 90 |
| Ex. No. 2-1 | 27 | 1 | 37 | 0 | 35 | 400 | 200 | 100 |
| Ex. No. 2-2 | 35 | 0.5 | 0 | 1 | 63.5 | 400 | 200 | 100 |
| Ex. No. 3 | 27 | 1 | 37 | 0 | 35 | 400 | 200 | 100 |
| Ex. No. 4-1 | 27 | 1 | 37 | 0 | 35 | 400 | 200 | 100 |
| Ex. No. 4-2 | 35 | 0.5 | 0 | 1 | 63.5 | 400 | 200 | 100 |
| Ex. No. 5 | 27 | 1 | 37 | 0 | 35 | 400 | 200 | 100 |
| Ex. No. 6-1 | 27 | 1 | 37 | 0 | 35 | 400 | 200 | 100 |
| Ex. No. 6-2 | 35 | 0.5 | 0 | 1 | 63.5 | 400 | 200 | 100 |
| Ex. No. 7 | 35 | 0.5 | 0 | 1 | 63.5 | 400 | 200 | 100 |
| Ex. No. 8 | 35 | 0.5 | 0 | 1 | 63.5 | 400 | 200 | 100 |
| Ex. No. 9 | 20 | 20 | 30 | 0 | 30 | 400 | 200 | 100 |
| Ex. No. 10 | 20 | 20 | 0 | 1 | 59 | 400 | 200 | 100 |
| Comp. Ex. No. 1 | 175 | 0 | 200 | 0 | 125 | 125 | 250 | 120 |
| Comp. Ex. No. | 540 | 0 | 740 | 0 | 720 | 2700 | 200 | 100 |
| 2-1 | | | | | | | | |
| Comp. Ex. No. | 35 | 0 | 0 | 1 | 64 | 200 | 200 | 100 |
| 2-2 | | | | | | | | |
| Comp. Ex. No. 3 | 540 | 0 | 740 | 0 | 720 | 2700 | 200 | 100 |
| Comp. Ex. No. 4 | 10 | 0 | 20 | 0 | 70 | 200 | 200 | 100 |
| Comp. Ex. No. | 10 | 0 | 20 | 0 | 70 | 200 | 200 | 100 |
| 5-1 | | | | | | | | |
| Comp. Ex. No. | 20 | 0 | 0 | 1 | 79 | 200 | 200 | 100 |
| 5-2 | | | | | | | | |
| Comp. Ex. No. 6 | 20 | 0 | 40 | 0 | 40 | 400 | 200 | 100 |
| Comp. Ex. No. | 20 | 0 | 40 | 0 | 40 | 400 | 200 | 100 |
| 7-1 | | | | | | | | |
| Comp. Ex. No. | 25 | 0 | 0 | 1 | 74 | 200 | 200 | 100 |
| 7-2 | | | | | | | | |
| Comp. Ex. No. 8 | 20 | 0 | 40 | 0 | 40 | 400 | 200 | 100 |
| Comp. Ex. No. | 2 | 0 | 35 | 0 | 63 | 400 | 200 | 100 |
| 9-1 | | | | | | | | |
| Comp. Ex. No. | 2.5 | 0 | 0 | 1 | 96.5 | 200 | 200 | 100 |
| 9-2 | | | | | | | | |

TABLE 2

| | Loading | Loading | Functional |
|---|---|---|---|
| | Component A | Component B | Ingredient |
| Ex. No. | Ascorbic Acid | 90%-by-mass | Colloidal Silica |
| 1 | | Purity Tea | |
| | | Catechin | |
| Ex. No. | Ascorbic Acid | Sodium | Colloidal Silica |
| 2-1 | | Hyaluronate | |
| Ex. No. | Ascorbic Acid | Sodium | Cellulose |
| 2-2 | | Hyaluronate | Acetate |
| Ex. No. | Ascorbic Acid | Collagen Peptide | Colloidal Silica |
| 3 | | | |
| Ex. No. | Ascorbic Acid | N-acetylglucosamine | Colloidal Silica |
| 4-1 | | | |
| Ex. No. | Ascorbic Acid | N-acetylglucosamine | Cellulose |
| 4-2 | | | Acetate |
| Ex. No. | Ascorbic Acid | Pyrrolidone | Colloidal Silica |
| 5 | | Carboxylic acid | |
| Ex. No. | Ascorbic Acid | Cysteine | Colloidal Silica |
| 6-1 | | | |
| Ex. No. | Ascorbic Acid | Cysteine | Cellulose |
| 6-2 | | | Acetate |
| Ex. No. | N-acetylglucosamine | Sodium | Cellulose |
| 7 | | Hyaluronate | Acetate |
| Ex. No. | Ascorbil | Sodium | Cellulose |
| 8 | Magnesium | Hyaluronate | Acetate |
| | Phosphate | | |
| Ex. No. | Ascorbic Acid | Tannic Acid | Colloidal Silica |
| 9 | | | |
| Ex. No. | Cysteine | Pyrrolidone | Cellulose |
| 10 | | Carboxylic acid | Acetate |
| Comp. | 90%-by-mass | None | Colloidal Silica |
| Ex. No. | Purity Tea | | |
| 1 | Catechin | | |
| Comp. | Ascorbic Acid | None | Colloidal Silica |
| Ex. No. | | | |
| 2-1 | | | |
| Comp. | Ascorbic Acid | None | Cellulose |
| Ex. No. | | | Acetate |
| 2-2 | | | |
| Comp. | Ascorbil | None | Colloidal Silica |
| Ex. No. | Magnesium | | |
| 3 | Phosphate (PAM) | | |
| Comp. | Collagen Peptide | None | Colloidal Silica |
| Ex. No. | | | |
| 4 | | | |
| Comp. | N-acetylglucosamine | None | Colloidal Silica |
| Ex. No. | | | |
| 5-1 | | | |
| Comp. | N-acetylglucosamine | None | Cellulose |
| Ex. No. | | | Acetate |
| 5-2 | | | |
| Comp. | Pyrrolidone | None | Colloidal Silica |
| Ex. No. | Carboxylic acid | | |
| 6 | | | |
| Comp. | Cysteine | None | Colloidal Silica |
| Ex. No. | | | |
| 7-1 | | | |
| Comp. | Cysteine | None | Cellulose |
| Ex. No. | | | Acetate |
| 7-2 | | | |
| Comp. | Tannic Acid | None | Colloidal Silica |
| Ex. No. | | | |
| 8 | | | |
| Comp. | Sodium | None | Colloidal Silica |
| Ex. No. | Hyaluronate | | |
| 9-1 | | | |
| Comp. | Sodium | None | Cellulose |
| Ex. No. | Hyaluronate | | Acetate |
| 9-2 | | | |

TABLE 3

| | Functional Component |
|---|---|
| Comp. Ex. No. 10 | Ascorbic Acid |
| Comp. Ex. No. 11 | 90%-by-mass Purity Tea Catechin |

[IR Measurement]

With respect to the test samples of Example No. 1, Comparative Example Nos. 1, 2-1, 10 and 11, the measurement of IR spectrum was done. (Using “JASCO FT/IR-5300,” the KBr Method)

The OH stretching vibrations at around wave numbers of 4,000-3,000 cm⁻¹, which were appreciated in the test samples of Comparative Example No. 10 (ascorbic acid) and Comparative Example No. 11 (90%-by-mass purity tea catechin), were decreased in the test samples of Example No. 1 and Comparative Example Nos. 1 and 2-1, in which they were loaded on the functional ingredient (colloidal silica). Moreover, in the test samples of Example No. 1 and Comparative Example Nos. 1 and 2-1, since a large peak was appreciated at around a wave number of 1,115 cm⁻¹, compared with Comparative Example Nos. 10 and 11, it was assumed that the Si—O—R bond generated. Presumably, due to the chemical reaction between colloidal silica and ascorbic acid (Example No. 1 and Comparative Example No. 2-1) or between colloidal silica and 90%-by-mass purity tea catechin (Example No. 1 and Comparative Example No. 1), it can be assumed that the Si—O—R bond generates.

Therefore, it is assumed that the test samples of Example No. 1, and the like, are not the simple mixtures of ascorbic acid, catechin and colloidal silica but the loading components and the functional ingredient (colloidal silica) undergo chemical bonds.

[ESR Measurement]

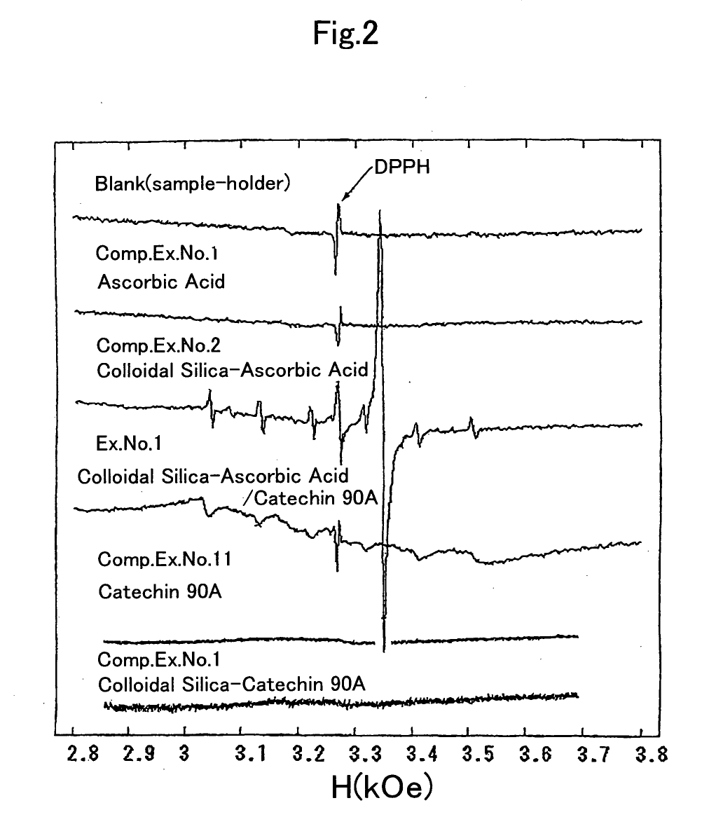

With respect to the test samples of Example No. 1, Comparative Example Nos. 1, 2-1, 10 and 11, the ESR spectra were measured. The results are illustrated in FIG. 2.

Comparing the ESR spectrum of the test sample of Comparative Example No. 2-1 in which only ascorbic acid was loaded on colloidal silica with the ESR spectrum of the test sample of Example No. 1 in which two types of loading components (ascorbic acid and 90%-by-mass purity tea catechin) were loaded on colloidal silica, since the ESR spectrum of Example No. 1 is broader, it is possible to assume that the mobility of existing radicals has fallen. Therefore, in the test sample of present Example No. 1, it was supposed that interactions, such as the generation of certain chemical bonds, arose between loaded catechin and ascorbic acid.

[Thermal Analysis Measurement]

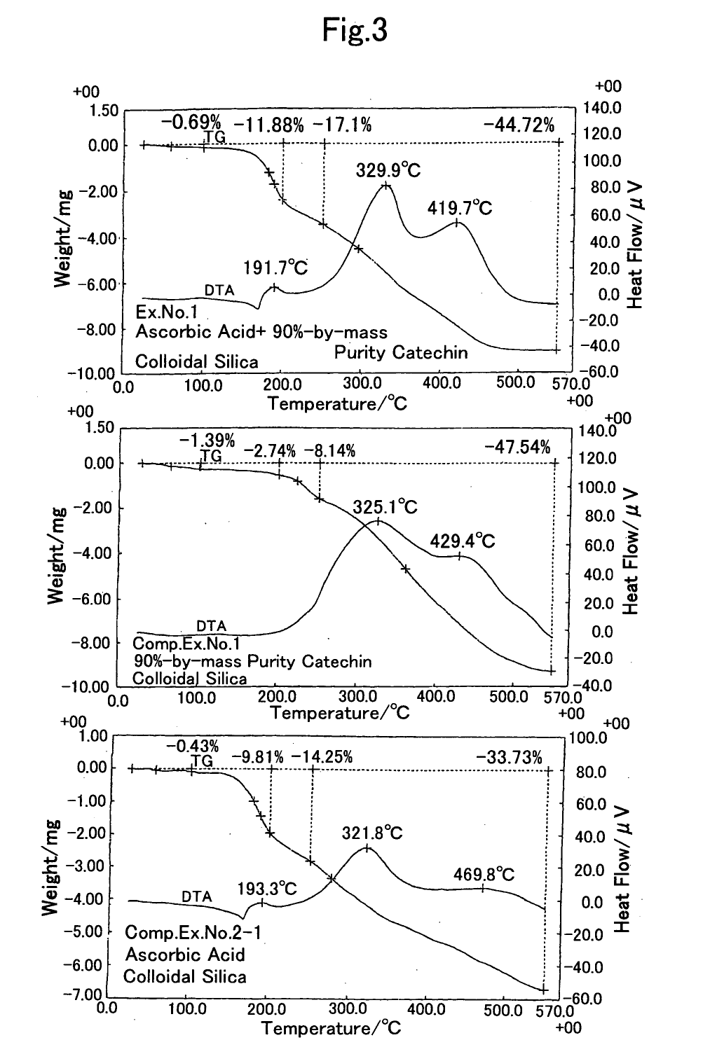

With respect to Example No. 1 in which 90%-by-mass purity tea catechin and ascorbic acid were employed as the loading components A and B and were combined with colloidal silica as the functional ingredient, a thermal analysis was carried out using DSC. (Temperature at Measurement Start 20° C., Temperature at Measurement Completion 500° C., Temperature Increment Rate 5° C./minute) And, with respect to Comparative Example Nos. 1 (Loading Component A) and 2-1 (Loading Component B) in which the loading components A and B, possessed by the test sample of Example No. 1, were loaded independently, respectively, a thermal analysis was carried out similarly using DSC. The results are illustrated in FIG. 3.

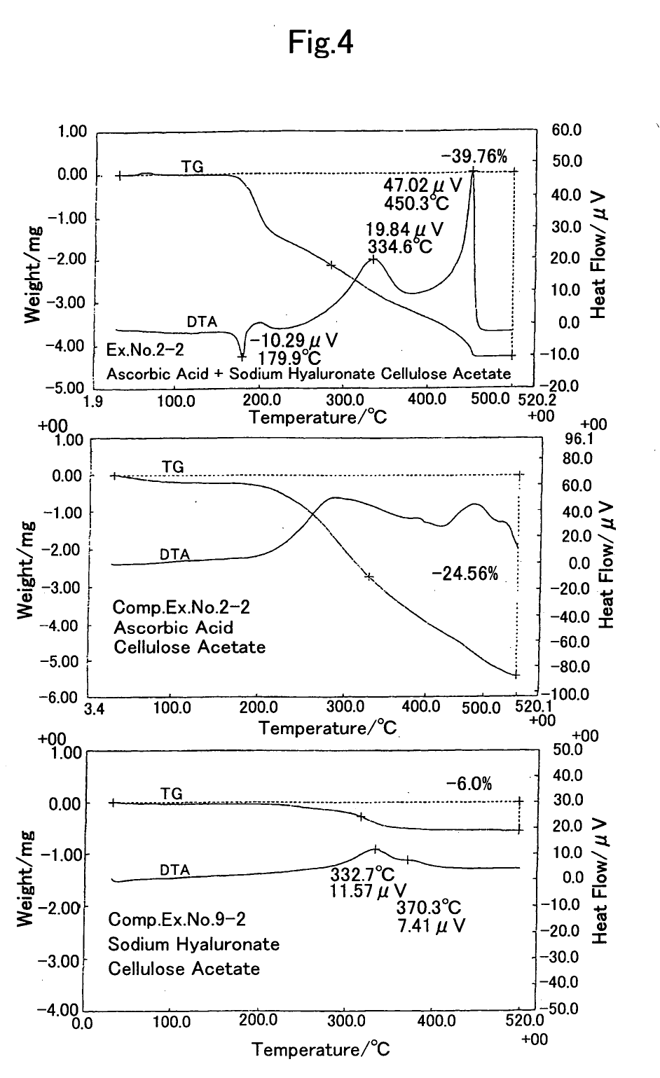

Moreover, with respect to Example No. 2-2 in which ascorbic acid and sodium hyaluronate were employed as the loading components A and B and were combined with cellulose acetate as the functional ingredient, a thermal analysis was carried out using DSC. (Temperature at Measurement Start 20° C., Temperature at Measurement Completion 500° C., Temperature Increment Rate 5° C./minute) And, with respect to Comparative Example Nos. 2-2 (Loading Component A) and 9-2 (Loading Component B) in which the loading components A and B, possessed by the test sample of Example No. 2-2, were loaded independently, respectively, a thermal analysis was carried out similarly using DSC. The results are illustrated in FIG. 4.

As a result, it became apparent that the thermal analysis results of the test samples of the respective examples were different from the simple superimpositions of the thermal analysis results of the test samples of the comparative examples. Therefore, it was suggested that the test samples of the examples were such that substances, which were different from the test samples of the comparative examples, were made.

[SEM Observation]

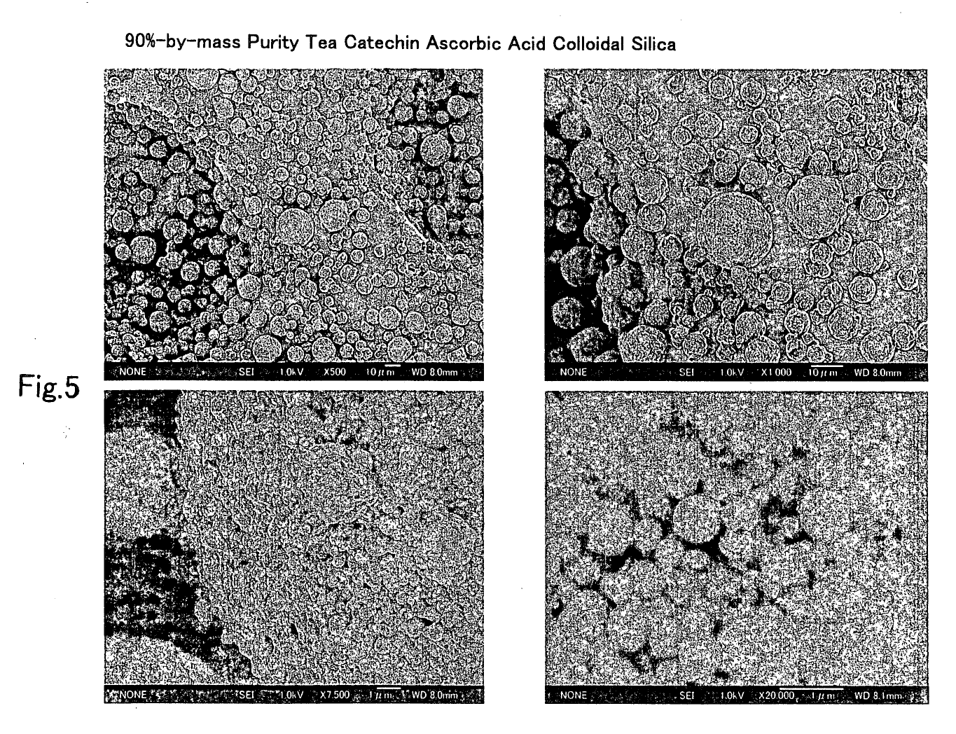

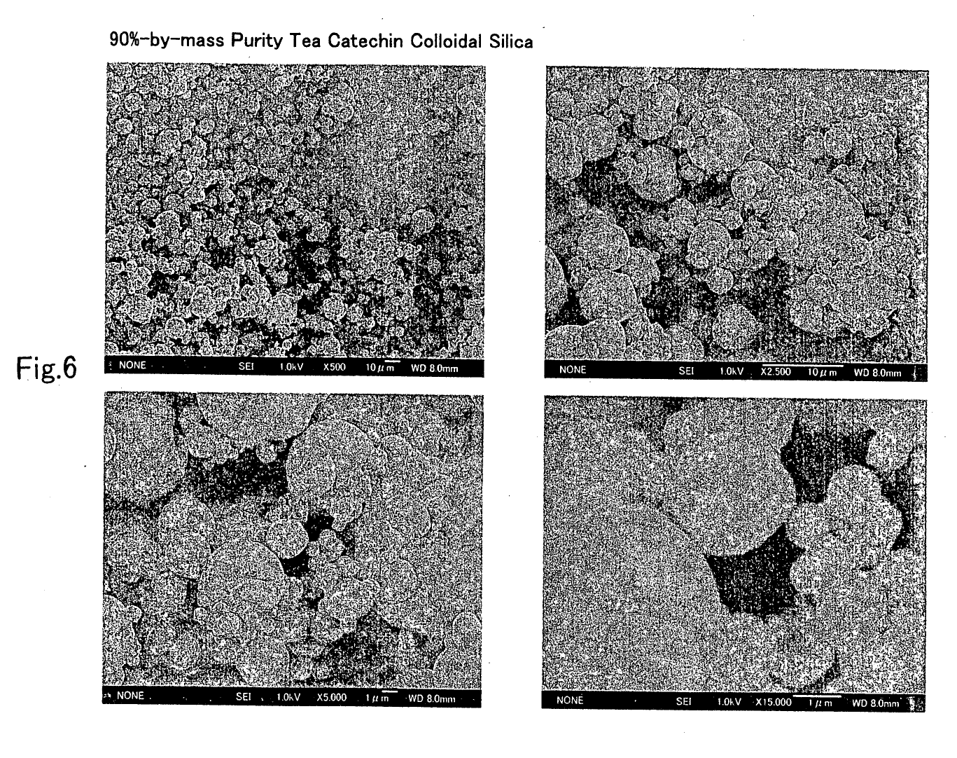

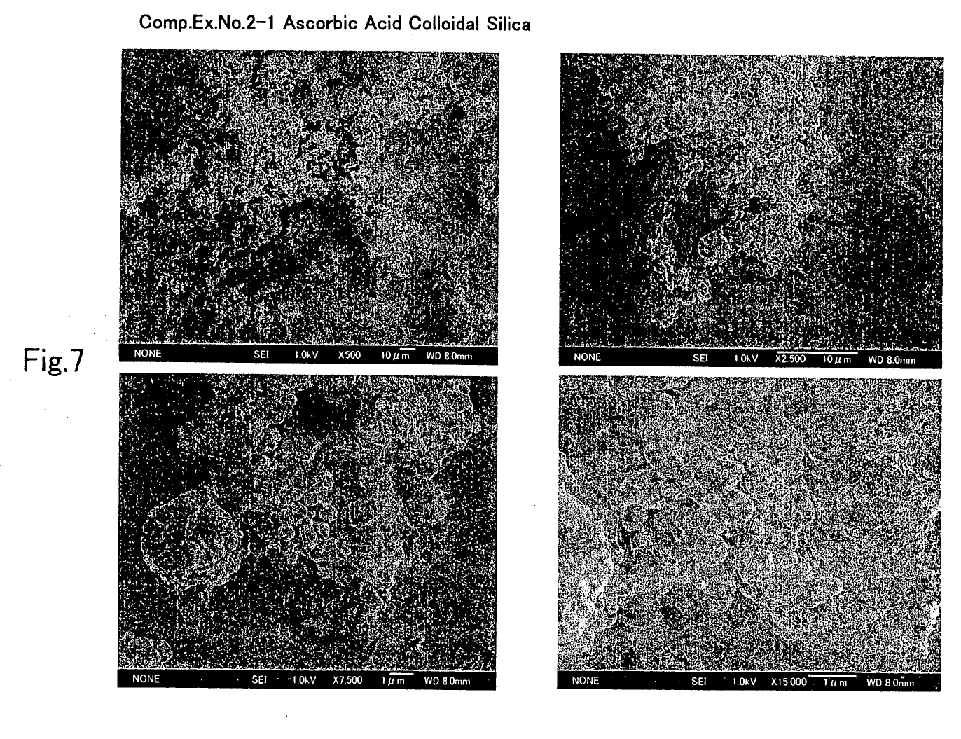

Using the samples of Example No. 1, Comparative Example No. 1 and Comparative Example No. 2-1, an SEM observation was carried out. In FIGS. 5, 6 and 7, the observation results of Example No. 1, Comparative Example No. 1 and Comparative Example No. 2-1 are shown.

All of the three samples were observed that, though the same colloidal silica was used as for the functional ingredient, the particle diameters, superficial shapes, and the like, of the obtained functional materials differed. In particular, Example No. 1 was such that, compared with Comparative Example Nos. 1 and 2-1, the appearance that the superficial shapes became more complete spherical shape was observed.

[Anti-Oxidation Ability Measurement]

Using the test samples of Example No. 1, Comparative Example Nos. 1, 2-1, 10 and 11, an anti-oxidation ability measurement was carried out.

The test samples of the respective example and comparative examples were coated on a honeycomb-shaped substrate. In a 25° C.-and-55%-moisture constant-temperature-and-constant-moisture chamber, air blasting was carried out at 1.1 m³/minute, with respect to the honeycomb-shaped substrate (dimension 50 mm Φ×10 mm T) with the test samples of the respective example and comparative examples coated, using a hair drier, and the air, which passed through the honeycomb-shaped substrates, was passed through pure water, thereby eluting the functional components (ascorbic acid and 90%-by-mass purity tea catechin), included in the air, into the pure water.

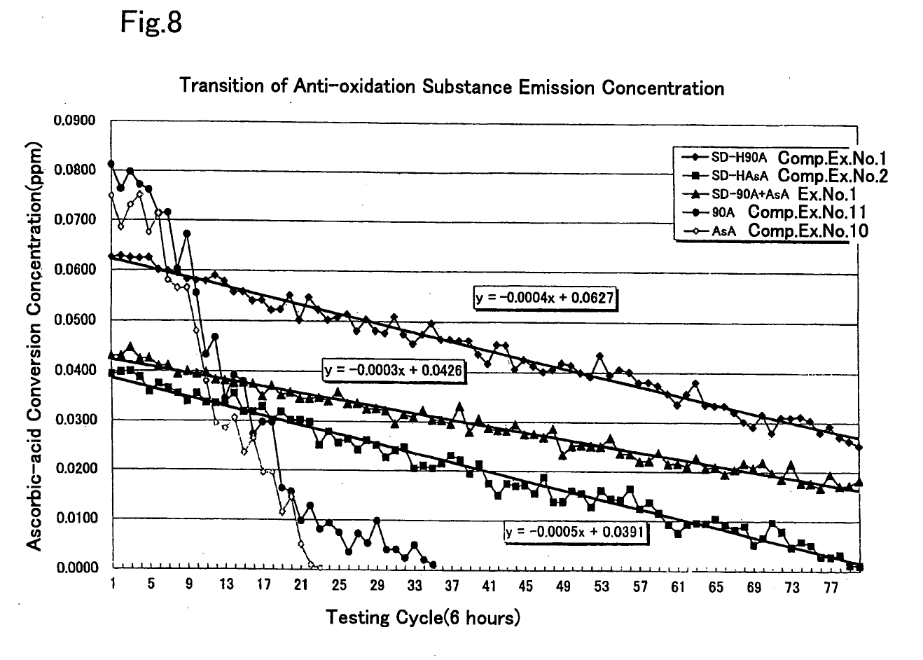

The concentrations of the anti-oxidation substances eluted into the liquid were measured using a “DPPH radical elimination method.” The “DPPH radical elimination method” is a method which utilizes the fact that an ethanol solution of DPPH (1,1-diphenyl-2-picryl hydrazile) discolors by the decrease of radicals to measure the amount of radicals, which are decreased by reacting the ethanol solution of DPPH with the pure water into which the functional components are eluted, by a spectrophotometer. The decreasing values of radical contents, which were caused when the functional components (ascorbic acid, and the like) reacted with DPPH, were represented as values for specifying anti-oxidation abilities. This time, using a working curve which was obtained from an ascorbic acid aqueous solution with a known concentration, the masses of the anti-oxidation substances were converted into the ascorbic acid concentrations to compare. A cycle test for every 6 hours was carried out 80 times, and the emission amounts of the anti-oxidation substances were plotted for every cycle. The graph of the test-cycle-number dependencies of the ascorbic-acid-conversion concentrations is illustrated in FIG. 8.

As it is apparent in FIG. 8, it became apparent that the emission amount of the anti-oxidation substances, which were emitted from the honeycomb-shaped substrate with the test sample of Example No. 1 coated was such that the gradient of the decreasing emission amount of the anti-oxidation substances was gentler, compared with the substrates with the test samples of Comparative Example No. 1 and Comparative Example No. 2-1 coated. Therefore, it was understood that the honeycomb-shaped substrate with the test sample of Example No. 1 coated was such that the longevity of the ability of emitting the anti-oxidation substances was longer, compared with the substrates with the test samples of Comparative Example No. 1 and Comparative Example No. 2-1 coated.

(Test #2)

The loading component was added to water, and was stirred until solids disappeared. Those which can dissolve into water dissolved completely. To this, silica fine particles whose average particle diameter was 1.5 μm (“ZEOSEAL® 1100V” produced by TAKI KAGAKU Co., Ltd.) was added, and was stirred until solids disappeared, and subsequently a solution, in which cellulose acetate (“CELL FLOW® TA-25” produced by CHISSO Co., Ltd.) or cellulose (“CELL FLOW® C-25” produced by CHISSO Co., Ltd.) was dispersed in a trace amount of ethanol, was added thereto and stirred, thereby preparing an aqueous slurry. The used cellulose acetate and cellulose were porous spherical fine particles which were close to complete sphere.

Moreover, those which used greenery alcohol for the functional component, slurries were prepared in the following procedures.

Ethanol was added to sepiolite, and was stirred until solids disappeared. To this, greenery alcohol was added, and was stirred. Subsequently, a solution, in which cellulose (“CELL FLOW® C-25” produced by CHISSO Co., Ltd.) was dispersed in a trace amount of ethanol, was added thereto and stirred, thereby preparing a slurry.

In Tables 4 and 5, there are set forth the mixing ratios of the respective contents, the hot-air set-up temperatures, and the touches of obtained functional materials. Moreover, regarding the types of the functional components and functional ingredients, they are set forth in Table 6. As for the functional components, tea-derived catechin was selected from the catechins to use; ascorbic acid was selected from the vitamins to use; sodium hyaluronate, cysteine (amino acid) and N-acetylglucosamine were selected from the natural moisturizing factors to use; and greenery alcohol was selected from the plants-derived essential oils to use.

Depending on the types of the functional components, cellulose and cellulose acetate, which are the functional ingredients, were used independently whenever appropriate.

As for an apparatus corresponding to FIG. 1, a spray-drying apparatus for research, “MICROMIST DRYER MDL-050-Type M” (one which was equipped with the quaternary-fluid nozzle) made by FUJISAKI DENKI Co., Ltd. was utilized, and the dropletizing step and drying step were carried out with respect to the aqueous slurries, and thereby producing-collecting the functional materials.

Those which used cellulose acetate and cellulose as the functional ingredient were labeled Example Nos. 11 through 20, those which used colloidal silica as the functional ingredient were labeled Comparative Example Nos. 10 through 12, and they were adapted to test samples, respectively. When colloidal silica was used instead of cellulose acetate or cellulose, a colloidal silica-40% water suspension (“ADELITE® AT-40” produced by ASAHI DENKA KOGYOU Co., Ltd.) was added in compositions set forth in Tables 4 and 5. Moreover, an ascorbic-acid simple substance, which is a functional component, was labeled Comparative Example No. 13, and was adapted to a test sample.

The functional materials obtained as the examples were fine particles whose touches were smooth and silky. Comparative Example Nos. 10 and 12 were such that the touches were rough, compared with the examples. It is believed to be due to the compounded colloidal silica. Moreover, Comparative Example No. 11, which was made by lowering the hot-air temperatures, was hardly dried, and no powder was obtained.

TABLE 4

| | | | Collodial | | | | | |
|---|---|---|---|---|---|---|---|---|
| | | Cellulose | Silica-40% | Silica | | | Hot-air | |
| | Functional | Acetate or | Water | Fine | | Hot-air | Outlet | Touch of |
| | Component | Cellulose | Suspension | Particles | Water | Inlet Set- | Set-up | Obtained |
| | (Parts | (Parts by | (Parts by | (Parts by | (Parts by | up Temp. | Temp. | Functional |
| | by Mass) | Mass) | Mass) | Mass) | Mass) | (° C.) | (° C.) | Material |
| Ex. No. 11 | 35 | 1 | 0 | 64 | 200 | 200 | 100 | Smooth and |
| | | | | | | | | Silky |
| Ex. No. 12 | 35 | 1 | 0 | 64 | 200 | 100 | 70 | Smooth and |
| | | | | | | | | Silky |
| Ex. No. 13 | 20 | 1 | 0 | 79 | 200 | 200 | 100 | Smooth and |
| | | | | | | | | Silky |
| Ex. No. 14 | 25 | 1 | 0 | 74 | 200 | 200 | 100 | Smooth and |
| | | | | | | | | Silky |
| Ex. No. 15 | 2.5 | 1 | 0 | 96.5 | 200 | 200 | 100 | Smooth and |
| | | | | | | | | Silky |
| Ex. No. 16 | 45 | 1 | 0 | 54 | 200 | 250 | 120 | Smooth and |
| | | | | | | | | Silky |
| Ex. No. 17 | 2.5 | 1 | 0 | 96.5 | 200 | 200 | 100 | Smooth and |
| | | | | | | | | Silky |
| Comp. Ex. | 540 | 0 | 740 | 720 | 2700 | 200 | 100 | Slightly |
| No. 10 | | | | | | | | Rough |
| Comp. Ex. | 540 | 0 | 740 | 720 | 2700 | 100 | 70 | No Powder |
| No. 11 | | | | | | | | Obtained |

TABLE 5

| | | | Colloidal | | | | | |
|---|---|---|---|---|---|---|---|---|
| | | Cellulose | Silica-40% | | | | Hot-air | |
| | Functional | Acetate or | Water | | | Hot-air | Outlet | Touch of |
| | Component | Cellulose | Suspension | Sepiolite | Ethanol | Inlet Set- | Set-up | Obtained |
| | (Parts | (Parts by | (Parts by | (Parts by | (Parts by | up Temp. | Temp. | Functional |
| | by Mass) | Mass) | Mass) | Mass) | Mass) | (° C.) | (° C.) | Material |
| Ex. No. 18 | 10 | 2 | 0 | 88 | 300 | 150 | 100 | Smooth and |
| | | | | | | | | Silky |
| Ex. No. 19 | 20 | 2 | 0 | 78 | 300 | 180 | 100 | Smooth and |
| | | | | | | | | Silky |
| Ex. No. 20 | 20 | 2 | 0 | 78 | 300 | 120 | 90 | Smooth and |
| | | | | | | | | Silky |
| Comp. Ex. | 10 | 0 | 20 | 70 | 300 | 180 | 100 | Slightly |
| No. 12 | | | | | | | | Rough |

TABLE 6

| | Functional Component | Functional Ingredient |
|---|---|---|
| Ex. No. 11 | Ascorbic Acid | Cellulose Acetate |
| Ex. No. 12 | Ascorbic Acid | Cellulose Acetate |
| Ex. No. 13 | N-acetylglucosamine | Cellulose Acetate |
| Ex. No. 14 | Cysteine | Cellulose Acetate |
| Ex. No. 15 | Sodium Hyaluronate | Cellulose Acetate |
| Ex. No. 16 | 90% by-mass Purity Tea | Cellulose Acetate |
| | Catechin | |
| Ex. No. 17 | Sodium Hyaluronate | Cellulose |
| Ex. No. 18 | Greenery Alcohol | Cellulose |
| Ex. No. 19 | Greenery Alcohol | Cellulose |
| Ex. No. 20 | Greenery Alcohol | Cellulose |
| Comp. Ex. No. 10 | Ascorbic Acid | Colloidal Silica |
| Comp. Ex. No. 11 | Ascorbic Acid | Colloidal Silica |
| Comp. Ex. No. 12 | Greenery Alcohol | Colloidal Silica |
| Comp. Ex. No. 13 | Ascorbic Acid | None |

[Thermal Analysis Measurement]

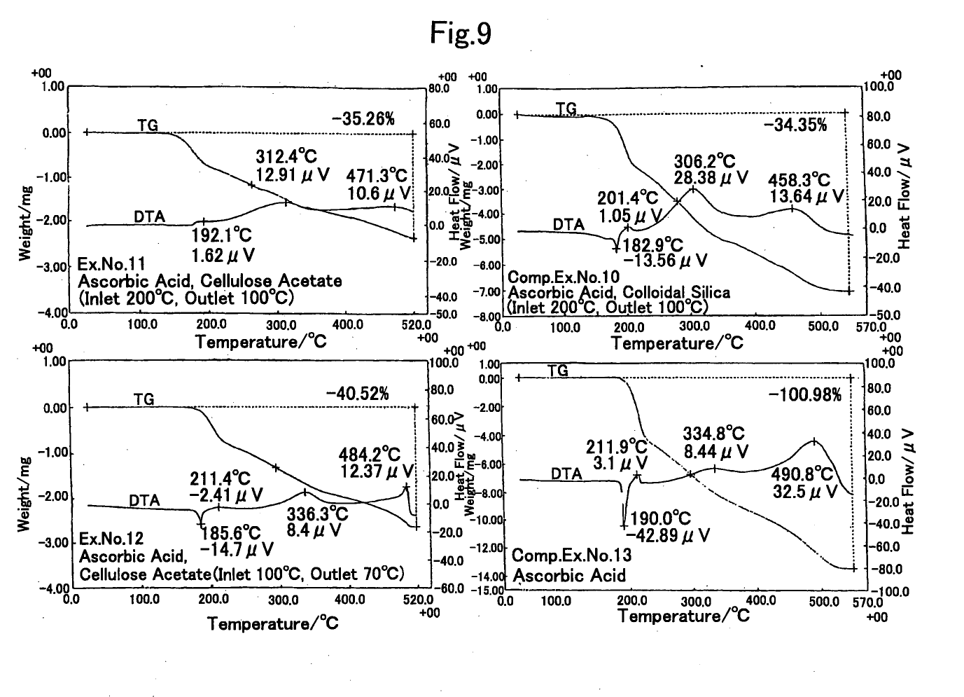

With respect to Example No. 11 in which ascorbic acid was employed as the functional component and in which cellulose acetate and ceramic fine particles were combined as the functional ingredient, a thermal analysis was carried out using DSC. (Temperature at Measurement Start 20° C., Temperature at Measurement Completion 500° C., Temperature Increment Rate 5° C./minute) And, with respect to Example No. 12 which lowered the production temperature of Example No. 11, moreover, Comparative Example No. 10 which changed the functional ingredient of Example No. 11 to colloidal silica and Comparative Example No. 13 which comprised ascorbic acid alone, a thermal analysis was carried out similarly using DSC. The results are illustrated in FIG. 9.

The DTA peak of the ascorbic-acid simple substance of Comparative Example No. 13 was seen, and the peak was also seen at the substantially same positions in the results of Example Nos. 11 and 12 and Comparative Example No. 10, and it is understood that each of them was loaded with ascorbic acid. Moreover, the minus % indications designated at the upper right of the respective results represent the weight reductions of the samples. It is believed that organic substances were burned to disappear so that they underwent the weight reductions. Comparative Example No. 13 became −100.98%, Example No. 11 became −35.26%, Example No. 12 became −40.52%, and Comparative Example No. 10 became −34.35%, and it was understood from the % s that the ascorbic acid and cellulose acetate, which were compounded organic substances, were included by 100% approximately in the samples.

[Anti-Oxidation Ability Measurement]

Using the test samples of Example No. 11, Example No. 12 and Comparative Example No. 10, an anti-oxidation ability measurement was carried out.

The anti-oxidation abilities were measured using a “DPPH radical elimination method.” The “DPPH radical elimination method” is a method which utilizes the fact that an ethanol solution of DPPH (1,1-diphenyl-2-picryl hydrazile) discolors by the decrease of radicals to measure the amount of radicals, which are decreased by adding the functional materials to an ethanol solution of DPPH and reacting the eluted functional components with it, by a spectrophotometer. The decreasing values of radical contents, which were caused when the functional components (ascorbic acid, and the like) reacted with DPPH, were represented as values for specifying anti-oxidation abilities.

This time, using a working curve which was obtained from ascorbic acid with a known mass, the anti-oxidation abilities were converted into the ascorbic acid contents to compare. Example No. 11, Example No. 12 and Comparative Example No. 10, which were made using ascorbic acid with the same compounding proportions, were adapted to test samples, and the anti-oxidation abilities of the respective 1-mg test samples were represented to correspond to how many mg of ascorbic acid. The results are set forth in Table 7.

As it is apparent from the results of Table 7, it was understood that Example No. 12 whose production temperature was low could maintain the anti-oxidation ability about twofold higher, compared with Example No. 11 which underwent the same production method but differed in the production temperature alone.

It is believed that the decomposition of ascorbic acid was suppressed by the low-temperature production.

TABLE 7

| | | | Ascorbic-acid |
|---|---|---|---|
| | Functional | Temperatures | Conversion |
| | Ingredient | during Production | Content (mg) |
| Ex. No. 11 | Cellulose | Inlet: 200° C., Outlet: 100° C. | 0.231 |
| | Acetate | | |
| Ex. No. 12 | Cellulose | Inlet: 100° C., Outlet: 70° C. | 0.412 |
| | Acetate | | |
| Comp. Ex. | Colloidal | Inlet: 200° C., Outlet: 100° C. | 0.107 |
| No. 10 | Silica | | |

[Sensory Test]

Using the test samples of Example No. 18 and Comparative Example No. 12, a test was carried out, regarding the sustainablities of the functional components' smells. Three monitors were asked to judge them. Example No. 18 and Comparative Example No. 12 were such that the same amount of greenery alcohol was used but the functional ingredients and production temperatures differed. (See Table 5 and Table 6.) The three monitors were asked to take a sniff of the respective 10-g samples which were placed on a dish at the same hour. The dishes were left in air, and they were asked to judge the smell levels at the respective points of time in accordance with the 6-stage representation method of smell intensity in Table 8. The sensory-test results on the test samples of Example No. 18 and Comparative Example No. 12 are set forth in Table 9. As it is apparent in Table 9, Comparative Example No. 12 was such that the smell was hardly sensed 3 days after the production, on the other contrary, Example No. 18 was such that the smell was sensed even after 10 days since the production. It was understood that it is Example No. 18 that greenery alcohol was loaded within the functional material for a longer period of time, compared with Comparative Example No. 12. Since the decomposition and evaporation of greenery alcohol were suppressed by the fact that the production temperature could be decreased, and moreover since cellulose was used for the functional ingredient, it is believed that greenery alcohol was loaded for a longer period of time.

TABLE 8

| | Strength |
|---|---|
| 6-stage Method | Intensity |
| Smell-less | 0 |
| Although it is not identifiable what smell it is, it can be sensed | 1 |
| to such an extent that it is sensed barely slightly. (Detection | |
| Threshold Value) | |
| Weak smell being identifiable what smell it is (Recognition | 2 |
| Threshold Value) | |
| Smell being sensed with ease (Moderate Strength) | 3 |
| Strong Smell | 4 |
| Strong Smell to such an extent being unendurable | 5 |

TABLE 9

| | Comp. Ex. No. 12 | | | Ex. No. 18 | | |
|---|---|---|---|---|---|---|
| | Functional Ingredient: | | | Functional Ingredient: | | |
| | Colloidal Silica | | | Cellulose Temps. | | |
| | Temps. during Production: | | | during Production: | | |
| | 180° C. at Inlet and | | | 150° C. at Inlet and | | |
| | 100° C. at Outlet | | | 100° C. at Outlet | | |
| | Monitor | | | Monitor | Monitor | |
| | A | Monitor B | Monitor C | A | B | Monitor C |
| Start | 4 | 4 | 4 | 4 | 4 | 4 |
| 3 Days | 1 | 1 | 1 | 3 | 3 | 3 |
| 10 Days | 0 | 0 | 0 | 3 | 2 | 2 |

(Test #3)

(Preparation of Test Samples: Method in which Loading Component is Loaded after Producing Support)

From among the functional components, ascorbic acid and tea catechin (90%-by-mass purity catechin) were selected, and were adapted to the loading components. In addition to dissolving ascorbic acid in water completely, tea catechin was added thereto, and was stirred until it dissolved therein. To this, pulverized silica whose average particle diameter was 1.5 μm (“ZEOSEAL® 1100V” produced by TAKI KAGAKU Co., Ltd.) was added, and was stirred until solids disappeared, and subsequently a solution, in which a colloidal silica-40% water suspension (“ADELITE® AT-40” produced by ASAHI DENKA KOGYOU Co., Ltd.) or cellulose acetate (“CELL FLOW® TA-25” produced by CHISSO Co., Ltd.) was dispersed in a trace amount of ethanol, was added, and was stirred, thereby preparing an aqueous slurry.

Since the colloidal silica-40% water suspension as it is is likely to gelate, an ion-exchange treatment was carried out in advance, and it was stabilized by adjusting the pH. The pulverized silica was added for the purpose of further pulverizing the functional materials. Note that colloidal silica and pulverized silica correspond to the functional ingredient.

Utilizing a spray-drying apparatus for research, “MICROMIST DRYER MDL-050-Type M” (one which was equipped with the quaternary-fluid nozzle) made by FUJISAKI DENKI Co., Ltd., spray-drying was carried out with respect to the prepared slurries, and mixtures of the respective loading components and functional ingredients were obtained. It is assumed that at least a part of the functional components in the loading components was bonded chemically to the functional ingredients. The spray-drying conditions are also set forth in Table 10.

TABLE 10

| | | | Colloidal | | | | | |
|---|---|---|---|---|---|---|---|---|
| | | | Silica-40% | | | | Hot-air | Hot-air |
| | Ascorbic | Tea | Water | Cellulose | Silica Fine | | Inlet | Outlet |
| | Acid | Catechin | Suspension | Acetate | Particles | Water | Set-up | Set-up |
| | (Parts | (Parts by | (Parts by | (Parts by | (Parts by | (Parts | Temp. | Temp. |
| | by Mass) | Mass) | Mass) | Mass) | Mass) | by Mass) | (° C.) | (° C.) |
| Ex. No. 21 | 125 | 50 | 125 | 0 | 500 | 500 | 180 | 90 |
| Comp. Ex. | 175 | 0 | 200 | 0 | 125 | 125 | 250 | 120 |
| No. 14 | | | | | | | | |
| Comp. Ex. | 0 | 540 | 740 | 0 | 720 | 2700 | 200 | 100 |
| No. 15 | | | | | | | | |

(Anti-Oxidation Ability Measurement)

Using the test samples of Example No. 21, Comparative Example Nos. 14 and 15 as well as Comparative Example No. 6 (tea catechin per se) and Comparative Example No. 17 (ascorbic acid per se), an anti-oxidation ability measurement was carried out.

The test samples of the respective example and comparative examples were coated on a honeycomb-shaped substrate. In a 25° C.-and-55%-moisture constant-temperature-and-constant-moisture chamber, air blasting was carried out at 1.1 m³/minute, with respect to the honeycomb-shaped substrate (dimension 50 mm Φ×10 mm T) with the test samples of the respective example and comparative examples coated, using a hair drier, and the air, which passed through the honeycomb-shaped substrates, was passed through pure water, thereby eluting the functional components (ascorbic acid and 90%-by-mass purity catechin), Included in the air, into the pure water.

The concentrations of the anti-oxidation substances eluted into the liquid were measured using a “DPPH radical elimination method.” The “DPPH radical elimination method” is a method which utilizes the fact that an ethanol solution of DPPH (1,1-diphenyl-2-picryl hydrazile) discolors by the decrease of radicals to measure the amount of radicals, which are decreased by reacting the ethanol solution of DPPH with the pure water into which the functional components are eluted, by a spectrophotometer. The decreasing values of radical contents, which were caused when the functional components (ascorbic acid, and the like) reacted with DPPH, were represented as values for specifying anti-oxidation abilities.

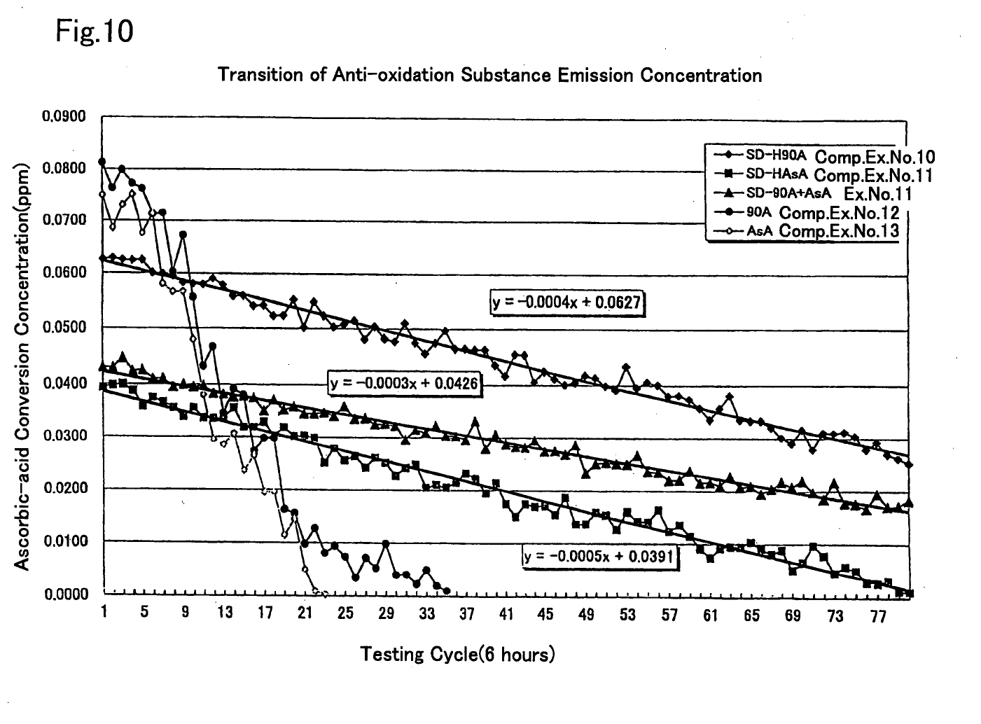

This time, using a working curve which was obtained from an ascorbic acid aqueous solution with a known concentration, the masses of the anti-oxidation substances were converted into the ascorbic acid concentrations to compare. A cycle test for every 6 hours was carried out 80 times, and the emission amounts of the anti-oxidation substances were plotted for every cycle. The graph of the test-cycle-number dependencies of the ascorbic-acid-conversion concentrations is illustrated in FIG. 10.

As it is apparent in FIG. 10, it became apparent that the emission amount of the anti-oxidation substances, which were emitted from the honeycomb-shaped substrate with the test sample of Example No. 21 coated was such that the gradient of the decreasing emission amount of the anti-oxidation substances was gentler, compared with the substrates with the test samples of Comparative Example No. 14 and Comparative Example No. 15 coated. Therefore, it was understood that the honeycomb-shaped substrate with the test sample of Example No. 14 coated was such that the longevity of the ability of emitting the anti-oxidation substances was longer, compared with the substrates with the test samples of Comparative Example No. 14 and Comparative Example No. 15 coated.

(On the Emission of Functional Component: On the Moisture Dependency of Ascorbic-Acid Emission Amount)

Using the test samples of Example No. 21, the emission amount of the functional member was measured. With respect to the honeycomb-shaped substrates (dimension 50 mm Φ×10 mm T) with the test sample of the example coated, air blasting was carried out at 4.5 L/minute, using 25-° C. air and with a hair drier. The moisture in the occasion was adapted to 55%, 65%, 75%, and 90%.

The air which passed through the honeycomb-shaped substrates was passed through water which was adjusted to pH 3, thereby eluting the functional components (ascorbic acid and 90%-by-mass purity catechin), included in the air, into the pure water.

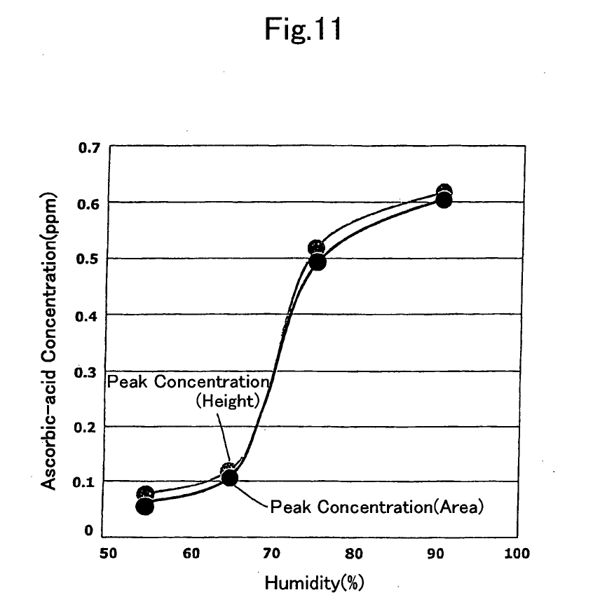

The emission amounts of the anti-oxidation substances eluted into the liquid were measured using the above-described “DPPH radical elimination method.” The emission amounts of the anti-oxidation substances were calculated by converting them into the concentrations of ascorbic acid. The results are illustrated in FIG. 11.

As it is apparent in FIG. 11, it was understood that the amount of ascorbic acid, which was emitted from the honeycomb-shaped substrate (the functional member of the present example) with the test sample coated increased rapidly when it was adapted to 75% or more (further 80% or more), compared with those when the humidity was up to 65%. That is, it was understood that the emission rate of the functional component was larger when high-humidity air was distributed than when low-humidity air was distributed.

(On the Stability of Functional Component: On the Stability of Ascorbic Acid)

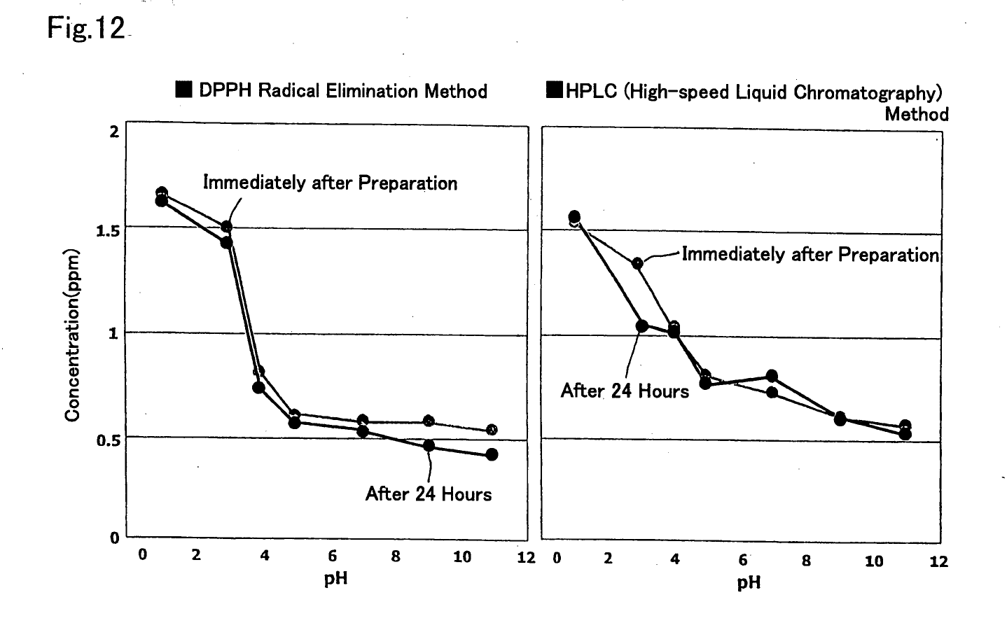

The free ascorbic-acid concentrations were measured immediately after they were prepared so that ascorbic acid was contained in an amount of 5 ppm in 25° C. water whose pH was adjusted to 1, 3, 4, 5, 7, 9 and 11, respectively, and after 24 hours. The free ascorbic acid concentrations were measured by the above-described DPPH radical elimination method and an HPLC method. The HPLC method uses an ODS as the column, and uses an ultraviolet spectrophotometer (wavelength 254 nm) as the detecting device to carry out the measurement. The results are illustrated in FIG. 12.

As it is apparent from FIG. 12, it became apparent that the lower the pH was the more the stability of ascorbic acid improved. In particular, it was understood that, with the boundary disposed at around a pH of 5, the stability improved when it was adapted to a pH of being this or less. Further, it was understood that it could be stabilized more when the pH was adapted to 3 or less.

(Preparation of Test Samples: Method of Loading Functional Component Simultaneously with the Production of Support)

Honeycomb bodies with loading components loaded were produced. The honeycomb bodies were molded in such a state that the loading components were mixed with the materials for producing the honeycomb bodies in advance, thereby producing the honeycomb bodies in which the loading components were contained simultaneously with the molding. Cordierite and sepiolite as the material constituting the support; green-tea catechin, ascorbic acid, magnesium ascorbate phosphate, H-hyaluronic acid, sodium hyaluronate, pyrrolidone sodium carboxylate and cystine as the functional components; and (colloidal) silica as the functional ingredient were used. In addition to them, a dispersant, a binding material, and the like, were added wherever appropriate. Regarding the main raw materials, the representative prescriptions are set forth in Table 11.

TABLE 11

| | Prescription | Prescription | Prescription | Prescription |
|---|---|---|---|---|
| Raw Material | #1 | #2 | #3 | #4 |
| Cordierite | 51 | 13 | 54 | 51 |
| Sepiolite | 0 | 48 | 6 | 15 |
| Green-tea | 0.4 | 3 | 2 | 0.4 |
| Catechin | | | | |
| Ascorbic Acid | 5 | 4 | 1 | 0.5 |
| Magnesium | 2 | 1 | 1 | 0.5 |
| Ascorbate | | | | |
| Phosphate | | | | |
| H-hyaluronic | 0 | 0 | 0 | 1 |
| Acid | | | | |
| Pyrrolidone | 0 | 0 | 0 | 1 |
| Sodium | | | | |
| Carboxylic | | | | |
| Acid | | | | |
| Cystine | 0 | 0 | 0 | 1 |
| Pulverized | 6 | 6 | 6 | 6 |
| Silica | | | | |
| Colloidal Silica | 10 | 10 | 10 | 10 |
| (Dried Mass | | | | |
| Conversion) | | | | |

Parts by Mass

Based on the above prescriptions, honeycomb-shaped functional members were produced by means of the following processes.

(1) Weighing and Compounding Powdery Raw Materials (Mortar Mixer)

The powdery raw materials (excepting the liquid-state substances such as colloidal silica and water) were weighed and mixed in their dry states.

(2) Kneading of Mixture Powders (Kneader, Vacuum Tug Mill, etc.)

While adding water and colloidal silica (40%-water suspension) to the aforementioned mixture powders little by little, they were wet kneaded. The addition amount of water was determined so as to be an appropriate property for extrusion molding described later.

(3) Extrusion Molding

Thereafter, in order to remove air included in the inside, honeycomb-shaped functional members were molded by an extrusion molding apparatus while further carrying out kneading in vacuum state.
(4) Drying-Calcining

After air drying the molded honeycomb-shaped functional members at ordinary temperature for 2-3 days, they were put into a calcination furnace. The temperature within the calcination furnace started from 110° C., and was adapted to a 10-° C./minute temperature increment rate. The maximum temperature was adapted to 600° C., and they were held at 600° C. for 1-1.5 hours. Thereafter, natural cooling was carried out. After cooling them to room temperature, they were cut to a predetermined size.

(Preparation of Test Samples: Method of Loading (Additionally Attaching) Functional Component after the Production of Support)

By repeating a cycle, in which a nonwoven cloth made of polyester was immersed into a composition liquid having the following composition and was then dried, for required cycles, a required amount of functional components was attached additionally on the surface. The composition liquid into which the nonwoven cloth was immersed was one in which, as the solid components, 1%-by-mass green-tea catechin, 4%-by-mass magnesium ascorbate phosphate, 5%-by-mass ascorbic acid, 1%-by-mass sodium hyaluronate, 84%-by-mass silica and 5%-by-mass anti-fungus agent were solved or suspended in water.

When air was passed through the prepared nonwoven cloth with the functional components loaded (functional member), it was confirmed by the DPPH radical elimination method that the functional components were emitted continuously.

(Test #4)

(Preparation of Test Samples: Spray-Drying Method (Method of the Present Invention))

A functional ingredient was added to water in which a functional component was solved, and was stirred until solids disappeared, thereby preparing an aqueous slurry. Wherever appropriate, a small amount of ethanol was added thereto to improve the dispersibility of the functional ingredient. When employing colloidal silica as the functional component, since it is likely to gelate as it is, an ion-exchange treatment was carried out in advance, and it was stabilized by adjusting the pH.

Utilizing a spray-drying apparatus for research, “MICROMIST DRYER MDL-050-Type M” (one which was equipped with the quaternary-fluid nozzle) made by FUJISAKI DENKI Co., Ltd., spray-drying was carried out, thereby obtaining functional materials of corresponding test examples, the mixtures of the respective loading components and functional ingredients. The compositions of the respective test examples and the spray-drying conditions are set forth in Table 12.

TABLE 12

| | | | | | Inlet | Outlet |
|---|---|---|---|---|---|---|
| | Functional Component | | Functional Ingredient | | Temp. | Temp. |
| Test | Catechin | 45 Parts | Colloidal | 20:35 | 250° C. | 120° C. |
| Example | | by Mass | Silica + Silica | | | |
| #1 | | | | | | |
| Test | | 35 | Cellulose | 65 | 200 | 100 |
| Example | | | | | | |
| #2 | | | | | | |
| Test | Ascorbic | 35 | Colloidal | 20:45 | 200 | 100 |
| Example | Acid | | Silica + Silica | | | |
| #3 | | | | | | |
| Test | | 35 | Cellulose | 65 | 200 | 100 |
| Example | | | | | | |
| #4 | | | | | | |
| Test | Hyaluronic | 2.5 | Colloidal | 17.7:79.5 | 200 | 100 |
| Example | Acid | | Silica + Silica | | | |
| #5 | | | | | | |
| Test | | 5 | Cellulose | 95 | 200 | 100 |
| Example | | | | | | |
| #6 | | | | | | |

(Preparation of Test Samples: Vat Method (Comparative Method))

A functional ingredient was added to water in which a functional component was solved, and was stirred until solids disappeared, thereby preparing an aqueous slurry. Wherever appropriate, a small amount of ethanol was added thereto to improve the dispersibility of the functional ingredient. When employing colloidal silica as the functional component, since it is likely to gelate as it is, an ion-exchange treatment was carried out in advance, and it was stabilized by adjusting the pH.

The obtained aqueous slurries were dried for a predetermined time within a drying furnace which was adjusted to a predetermined temperature. The compositions of the respective test examples and the drying conditions are set forth in Table 13.

TABLE 13

| | | | | | Drying | Drying |
|---|---|---|---|---|---|---|
| | Functional Component | | Functional Ingredient | | Temp. | Time. |
| Test | Catechin | 45 Parts | Colloidal | 20:35 | 150° C. | 8 hours |
| Example | | by Mass | Silica + Silica | | | |
| #7 | | | | | | |
| Test | | 35 | Cellulose | 65 | 120 | 5 |
| Example | | | | | | |
| #8 | | | | | | |
| Test | Ascorbic | 35 | Colloidal | 20:45 | 150 | 8 |
| Example | Acid | | Silica + Silica | | | |
| #9 | | | | | | |
| Test | | 35 | Cellulose | 65 | 120 | 5 |
| Example | | | | | | |
| #10 | | | | | | |
| Test | Hyaluronic | 2.5 | Colloidal | 17.7:79.5 | 150 | 8 |
| Example | Acid | | Silica + Silica | | | |
| #11 | | | | | | |
| Test | | 5 | Cellulose | 95 | 120 | 5 |
| Example | | | | | | |
| #12 | | | | | | |

(TG/DTA Measurement)

Regarding the test samples of the respective test examples, a TG/DTA measurement was carried out. As a result, the test samples produced by the spray-drying method and the test samples produced by the vat method had peaks at different positions and with different heights even for the test samples whose composition and compositional ratio of the functional ingredients and functional component were the same.

(Dispersion Test)

After weighing out the respective samples of Test Example Nos. 1 through 12 by 0.2 g in a 50-mL centrifugation tube, 20-mL linseed oil was added thereto and was stirred for 1 hour. Thereafter, they were left for 4 hours, and were inspected visually for the occurrence of precipitate generation and precipitate separation.

20-mL water was added to each of the centrifugation tubes, and was stirred. After 3 hours and 18 hours had passed since the mixing of water, 10-mL suspension was collected, and a centrifugal separation was carried out at 4,000 rpm for 10 minutes, and thereafter the water phase was collected to measure the concentration of the functional components included therein. The measurement of the concentrations was carried out by means of the DPPH method (as described above) for catechin and ascorbic acid and a carbazole sulfuric acid method for hyaluronic acid. The results are set forth in Table 14.

TABLE 14

| | Functional | Functional | Appearance of | After | After |
|---|---|---|---|---|---|
| | Component | Ingredient | Suspension | 3 hours | 18 hours |
| Test | Catechin | Colloidal | Separation | 63 | 72 |
| Example #1 | | Silica + Silica | | | |
| Test | | Cellulose | Suspension | 467 | 513 |
| Example #2 | | | | | |
| Test | Ascorbic Acid | Colloidal | Separation | 18 | 23 |
| Example #3 | | Silica + Silica | | | |
| Test | | Cellulose | Suspension | 197 | 212 |
| Example #4 | | | | | |
| Test | Hyaluronic | Colloidal | Separation | 35 | 42 |
| Example #5 | Acid | Silica + Silica | | | |
| Test | | Cellulose | Suspension | 305 | 330 |
| Example #6 | | | | | |
| Test | Catechin | Colloidal | Separation | 46 | 52 |
| Example #7 | | Silica + Silica | | | |
| Test | | Cellulose | Separation | 36 | 39 |
| Example #8 | | | | | |
| Test | Ascorbic Acid | Colloidal | Separation | 15 | 17 |
| Example #9 | | Silica + Silica | | | |
| Test | | Cellulose | Separation | 12 | 13 |
| Example #10 | | | | | |
| Test | Hyaluronic Acid | Colloidal | Separation | 26 | 25 |
| Example #11 | | Silica + Silica | | | |
| Test | | Cellulose | Separation | 22 | 35 |
| Example #12 | | | | | |

Units; μg/L

As it is apparent from Table 14, it was understood that Test Example Nos. 2, 4 and 6, which were prepared by the spray-drying method and employed cellulose alone as the functional ingredient, were good in terms of the dispersibility with respect to linseed oil, and at the same time could emit the functional components with respect to water quickly. Moreover, it was understood that Test Example Nos. 8, 10 and 12, which employed the vat method instead of the spray-drying method, were such that the dispersibility was low and the emission characteristic of the functional components with respect to water was not sufficient as well, though they had the same compositions as those of Test Example Nos. 2, 4 and 6.

(Sensory Test)

Regarding Test Example Nos. 5, 6, 11 and 12 which employed hyaluronic acid as the functional component, their sensitivity (rough feelings and smooth feelings) when they were rubbed into skin was evaluated in 5 stages. Specifically, the test samples were rubbed into subject's cheek and back of the hand to carry out the evaluation in a testing room which was adjusted to 25° C. room temperature and 50% relative humidity. The subjects had stayed in the testing room for 1 hour prior to it, and the evaluation was carried out after their skin conditions were stabilized.

The test samples were adapted to particle diameters of 3 μm or less with a ball mill and a jet mill. The subjects were adapted to 9 peoples (5 females: 22, 29, 35, 43 and 50 years old; and 4 males: 20, 27, 35 and 52 years old).

As a result, in both cheek and back of the hand, an outcome that the test samples of Test Example Nos. 11 and 12 (produced by the vat method) were not favorable was obtained, on the other hand, the test samples of Test Example Nos. 5 and 6, which were produced by the spray-drying method, gave good feelings. In particular, all the subjects judged that Test Example No. 6, which employed cellulose as the functional ingredient, was the best.

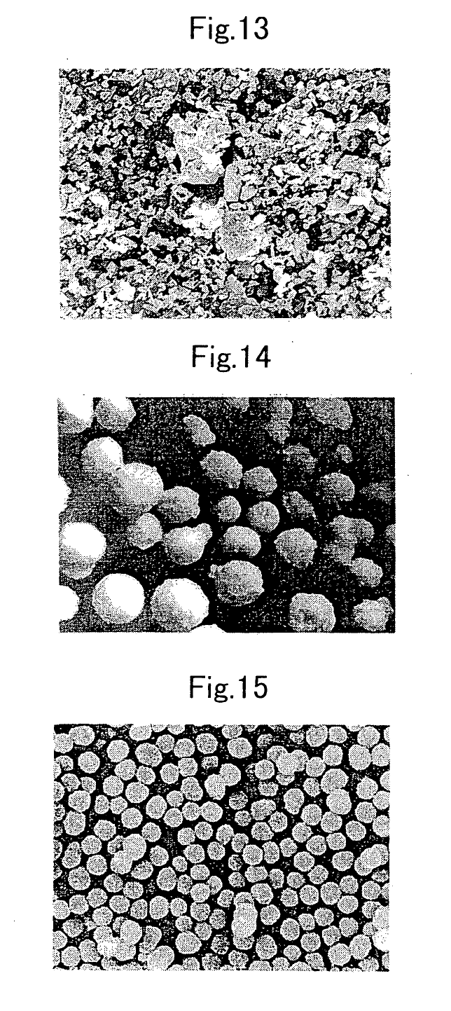

Therefore, when considering the results of the above-described dispersion test combinedly therewith, it was understood that the functional materials, which were produced by the spray-drying method being good in terms of the dispersibility with respect to linseed oil and which employed cellulose as the functional ingredient, gave favorable feelings with respect to skin. This is apparent from the electron microscopic pictures of Test Example No. 11 (FIG. 13: produced by the vat method) and Test Example Nos. 5 and 6 (FIGS. 14 and 15: produced by the spray-drying method). That is, Test Example No. 11, produced by the vat method, was such that the particulate surfaces were angulated, on the other hand, the particulate surfaces of Test Example Nos. 5 and 6, produced by the spray-drying method, were smooth. In particular, Test Example No. 6 had much smoother surfaces than the particulate surfaces of Test Example No. 5.

(Test #5)

(Preparation of Test Samples: Spray-Drying Method (Method of the Present Invention))

A functional ingredient was added to water in which a functional component was solved, and was stirred until solids disappeared, thereby preparing an aqueous slurry.

Utilizing a spray-drying apparatus for research, “MICROMIST DRYER MDL-050-Type M” (one which was equipped with the quaternary-fluid nozzle) made by FUJISAKI DENKI Co., Ltd., spray-drying was carried out, thereby obtaining functional materials of corresponding test examples, the mixtures of the respective loading components and functional ingredients. The compositions of the respective test examples and the spray-drying conditions are set forth in Table 15. Here, as for the water-soluble dietary fibers, natural water-soluble dietary fibers, and the like, which were obtained by subjecting gua bean to enzymatic decomposition and refinement, were used.

TABLE 15

| | Functional | Functional | | | Inlet | Outlet |
|---|---|---|---|---|---|---|
| | Component | Ingredient | Total | Water | Temp. | Temp. |
| Test | Catechin | Water- | | 1000 g | 200° C. | 100° C. |
| Example | 30A, | soluble | | | | |
| #13 | Theanine, | Dietary | | | | |
| | and Ascorbic | Fibers | | | | |
| | Acid | | | | | |
| | 50% | 50% | 100% | | | |
| | 100 g | 100 g | 200 g | | | |
| Test | Hyaluronic | Water- | | 1000 g | 200° C. | 100° C. |
| Example | Acid | soluble | | | | |
| #14 | | Dietary | | | | |
| | | Fibers | | | | |
| | 5.00% | 95% | 100% | | | |
| | 5 g | 95 g | 100 g | | | |

What is claimed is:

1. A functional material exhibiting dispersibility with respect to linseed oil, the functional material being characterized in that it has: a loading component selected from functional components comprising the group consisting of catechins, vitamins, tannins, natural moisturizing factors and essential oils being derived from plants; and
a functional ingredient being constituted of an organic polymer material in which the loading component is loaded on the surface or in the inside thereof, and which is a fine particle.

2. The functional material set forth in claim 1, being producible by a dropletizing step of turning an aqueous slurry having said loading component and said functional ingredient into a fine droplet state; and
a drying step of drying the fine droplet by contacting it with a hot air.

3. The functional material set forth in claim 1, wherein said functional ingredient is one or more organic polymer materials selected from the group consisting of organic polymer materials being derived from plants and animals, and their derivatives as well as polyvinyl alcohol.

4. The functional material set forth in claim 1, wherein it does not contain an inorganic component substantially.

5. A functional material, being characterized in that it has: two or more loading components selected from functional components comprising the group consisting of catechins, vitamins, tannins, natural moisturizing factors and essential oils being derived from plants, and at least a part thereof interacting with each other; and
a functional ingredient in which the loading component is loaded on the surface or in the inside thereof, and which is a fine particle.

6. The functional material set forth in claim 1, wherein said interactive action between said functional components possessed by said loading components is a chemical bond; and
at least a part of said loading components is loaded by chemically bonding with the surface or inside of said functional ingredient.

7. A functional material, being producible by a dropletizing step of turning an aqueous slurry having two or more loading components selected from functional components comprising the group consisting of catechins, vitamins, tannins, natural moisturizing factors and essential oils being derived from plants, and a functional ingredient being a fine particle into a fine droplet state; and
a drying step of drying the fine droplet by contacting it with a hot air.

8. The functional material set forth in claim 7, wherein a temperature of said hot air is such that:
an inlet temperature is 100° C. or more and 300° C. or less; and
an exhaust temperature is 65° C. or more and 250° C. or less, and is lower than the inlet temperature by 30° C. or more.

9. The functional material set forth in claim 1, wherein a mixing ratio of said loading component to said functional ingredient is such that the loading component is adapted to 1 part by mass or more and 70 parts by mass or less when said functional ingredient is taken as 100 parts by mass.

10. The functional material set forth in claim 1, wherein a mixing ratio of arbitrary two members among said functional components, which said loading component contains, is 1:100 or more and 100:1 or less on the mass basis.

11. The functional material set forth in claim 1, wherein said functional components and said functional ingredient have an OH group in their chemical structures.

12. The functional material set forth in claim 1, wherein an average particle diameter of said functional material is 20 μm or less.

13. The functional material set forth in claim 1, wherein said catechins are catechin being derived from tea.

14. The functional material set forth in claim 1, wherein said vitamins include at least one member selected from the group consisting of vitamin, vitamin derivatives, and vitamin-like substances acting like vitamin.

15. The functional material set forth in claim 1, wherein said tannins include at least one member selected from the group consisting of tannin, tannic acid, pyrogallol, gallic acid, and gallic ester.

16. The functional material set forth in claim 1, wherein said natural moisturizing factors include at least one member selected from the group consisting of hyaluronic acids comprising hyaluronic acid and its salts, amino acid, polyamino acid, pyrrolidone carboxylic acid and its salts, and N-acetylglucosamine, animal and plant polysaccharide, co-enzyme Q10, rice powders, gelatin, oligosaccharide, monosaccharides, saponins, vegetable peptide, phospholipid, sericin, chondroitin, ceramide, albumin, collagen, and chitin as well as chitosan.

17. The functional material set forth in claim 1, wherein said essential oils being derived from plants exhibit at least one property selected from the group consisting of anti-microorganism properties, deodorizing properties, anti-allergenic properties, anti-oxidation properties, anti-inflammation properties, relaxation properties, aroma-therapeutic properties, moisturizing properties, and noxious-minor-creature rejective actions.

18. The functional material set forth in claim 1, wherein said functional ingredient comprises one or more materials selected from the group consisting of inorganic ingredients comprising colloidal silica, calcium silicate, ethyl silicate, sodium silicate, potassium silicate, lithium silicate, calcium aluminate, β-alumina, boehmite, alumina sol, calcium phosphate, aluminum phosphate and magnesium phosphate, and organic ingredients comprising cellulose, cellulose acetate, carboxymethylcellulose and polyvinyl alcohol.

19. The functional material set forth in claim 1, wherein said loading component is green-tea catechin and ascorbic acid; and
said functional ingredient is colloidal silica.

20. The functional material set forth in claim 5, wherein said loading component is ascorbic acid and sodium hyaluronate; and
said functional ingredient is colloidal silica.

21. The functional material set forth in claim 1, wherein said loading component is ascorbic acid and sodium hyaluronate; and
said functional ingredient is cellulose acetate.

22. The functional material set forth in claim 5, wherein said loading component is ascorbic acid and collagen peptide; and
said functional ingredient is colloidal silica.

23. The functional material set forth in claim 5, wherein said loading component is ascorbic acid and N-acetylglucosamine; and
said functional ingredient is colloidal silica.

24. The functional material set forth in claim 1, wherein said loading component is ascorbic acid and N-acetylglucosamine; and
said functional ingredient is cellulose acetate.

25. The functional material set forth in claim 5, wherein said loading component is ascorbic acid and pyrrolidone carboxylic acid; and
said functional ingredient is colloidal silica.

26. The functional material set forth in claim 5, wherein said loading component is ascorbic acid and cystine; and
said functional ingredient is colloidal silica.

27. The functional material set forth in claim 1, wherein said loading component is ascorbic acid and cystine; and
said functional ingredient is cellulose acetate.

28. The functional material set forth in claim 1, wherein said loading component is ascorbic acid and pyrrolidone carboxylic acid; and
said functional ingredient is cellulose acetate.

29. The functional material set forth in claim 1, wherein said loading component is N-acetylglucosamine and sodium hyaluronate; and
said functional ingredient is cellulose acetate.

30. The functional material set forth in claim 1, wherein said loading component is magnesium ascorbate phosphate and sodium hyaluronate; and
said functional ingredient is cellulose acetate.

31. The functional material set forth in claim 5, wherein said loading component is ascorbic acid and tannic acid; and
said functional ingredient is colloidal silica.

32. The functional material set forth in claim 1, wherein said loading component is cystine and pyrrolidone carboxylic acid; and
said functional ingredient is cellulose acetate.

33. A process for producing a functional material, being characterized in that it has: a dropletizing step of turning an aqueous slurry having two or more loading components selected from the group consisting of catechins, vitamins, tannins, natural moisturizing factors and essential oils being derived from plants, and a functional ingredient being a fine particle into a fine droplet state; and
a drying step of drying the fine droplet by contacting it with a hot air.

34. A functional member, being characterized in that it has: two or more loading components selected from functional components comprising the group consisting of catechins, vitamins, tannins, natural moisturizing factors and essential oils, and at least a part thereof interacting with each other; and
a support loading the loading components thereon or containing them therein, and being capable of letting air pass in at least one direction.

35. The functional member set forth in claim 34, wherein said support is a porous body or a fiber aggregate.

36. The functional member set forth in claim 1, wherein said support is formed of nonwoven cloth and/or ceramic molded body.

37. The functional member set forth in claim 34, wherein said loading components include ascorbic acid; and
it further has a pH-adjusting agent for adjusting pH to 5 or less.

38. The functional member set forth in claim 34 having a particulate functional ingredient loaded on said support or contained in it, wherein
a part of said functional components of said loading components is bonded chemically to the functional ingredient.

39. An environment modifying apparatus being characterized in that it has a filter being the functional member set forth in claim 34, air-sending-out means for letting air pass through the filter, and moisturizing means for moisturizing the air passing through the filter.

40. A functional material, being characterized in that it has:
ceramic particles;
a functional ingredient selected from the group consisting of cellulose, cellulose derivatives and polyvinyl alcohol, and adhering on a surface of said ceramic particles or covering it;
a functional component selected from the group consisting of catechins, vitamins, tannins, natural moisturizing factors and essential oils being derived from plants, and contained inside said functional ingredient or loaded on a surface thereof.

41. A functional material, being characterized in that it is producible by a dropletizing step of turning an aqueous slurry into a fine droplet state, the aqueous slurry having:
ceramic particles;
a functional ingredient selected from the group consisting of cellulose, cellulose derivatives and polyvinyl alcohol; and
a functional component selected from the group consisting of catechins, vitamins, tannins, natural moisturizing factors and essential oils being derived from plants; and
a drying step of drying said fine droplet by contacting it with a hot air.

42. The functional material set forth in claim 41, wherein a temperature of said hot air is such that:
an inlet temperature is 100° C. or more and 300° C. or less; and
an exhaust temperature is 65° C. or more and 250° C. or less, and is lower than the inlet temperature by 30° C. or more.

43. The functional material set forth in claim 40, wherein said ceramic particles are silica fine particles.

44. The functional material set forth in claim 40, wherein an average particle diameter of said ceramic particles is 2 μm or less.

45. The functional material set forth in claim 40, wherein a mixing ratio of said functional component and said functional ingredient to said ceramic particles is such that said functional component is 1 parts by mass or more and 70 parts by mass or less, and said functional ingredient is 10 parts by mass or less, when said ceramic particles are taken as 100 parts by mass.

46. The functional material set forth in claim 40, wherein an average particle diameter of said functional material is 20 μm or less.

47. The functional material set forth in claim 40, wherein said catechins are catechin being derived from tea.

48. The functional material set forth in claim 40, wherein said vitamins include at least one member selected from the group consisting of vitamin, vitamin derivatives, and vitamin-like substances acting like vitamin.

49. The functional material set forth in claim 40, wherein said tannins include at least one member selected from the group consisting of tannin, tannic acid, pyrogallol, gallic acid, and gallic ester.

50. The functional material set forth in claim 40, wherein said natural moisturizing factors include at least one member selected from the group consisting of hyaluronic acids comprising hyaluronic acid and its salts, amino acid, polyamino acid, pyrrolidone carboxylic acid and its salts, and N-acetylglucosamine, animal and plant polysaccharide, co-enzyme Q10, rice powders, gelatin, oligosaccharide, monosaccharides, saponins, vegetable peptide, phospholipid, sericin, chondroitin, ceramide, albumin, collagen, and chitin as well as chitosan.

51. The functional material set forth in claim 40, wherein said essential oils being derived from plants exhibit at least one property selected from the group consisting of anti-microorganism properties, deodorizing properties, anti-allergenic properties, anti-oxidation properties, anti-inflammation properties, relaxation properties, aroma-therapeutic properties, moisturizing properties, and noxious-minor-creature rejective actions.

52. The functional material set forth in claim 40, wherein:
said functional component is ascorbic acid;
said functional ingredient is cellulose acetate; and said ceramic particles are silica fine particles.

53. The functional material set forth in claim 40, wherein:
said functional component is N-acetylglucosamine;
said functional ingredient is cellulose acetate; and said ceramic particles are silica fine particles.

54. The functional material set forth in claim 40, wherein:
said functional component is cystine;
said functional ingredient is cellulose acetate; and said ceramic particles are silica fine particles.

55. The functional material set forth in claim 40, wherein:
said functional component is sodium hyaluronate;
said functional ingredient is cellulose acetate; and said ceramic particles are silica fine particles.

56. The functional material set forth in claim 40, wherein:
said functional component is tea catechin;
said functional ingredient is cellulose acetate; and said ceramic particles are silica fine particles.

57. The functional material set forth in claim 40, wherein:
said functional component is sodium hyaluronate;
said functional ingredient is cellulose; and said ceramic particles are silica fine particles.

58. The functional material set forth in claim 40, wherein:
said functional component is greenery alcohol;
said functional ingredient is cellulose; and said ceramic particles are sepiolite.

59. A process for producing a functional material, being characterized in that it has:
a dropletizing step of turning an aqueous slurry into a fine droplet state, the aqueous slurry having:
ceramic particles;
said functional ingredient selected from the group consisting of cellulose, cellulose derivatives and polyvinyl alcohol; and
a functional component selected from the group consisting of catechins, vitamins, tannins, natural moisturizing factors and essential oils being derived from plants; and
a drying step of drying said fine droplet by contacting it with a hot air.
